SEQUENCE LISTING (7 sequences)

SEQ ID NO: 1 - protein, 384 aa, Azotobacter vinelandii
MASVIIDDTTLRDGEQSAGVAFNADEKIAIARALAELGVPELEIGIPSMGEEEREVMHAIAGLGLSSRLLAWCRLCDVDLAAARSTGVTMVDLSLPVSDLMLHHKLNRDRDWALREVARLVGEARMAGLEVCLGCEDASRADLEFVVQVGEVAQAAGARRLRFADTVGVMEPFGMLDRFRFLSRRLDMELEVHAHDDFGLATANTLAAVMGGATHINTTVNGLGERAGNAALEECVLALKNLHGIDTGIDTRGIPAISALVERASGRQVAWQKSVVGAGVFTHEAGIHVDGLLKHRRNYEGLNPDELGRSHSLVLGKHSGAHMVRNTYRDLGIELADWQSQALLGRIRAFSTRTKRSPQPAELQDFYRQLCEQGNPELAAGGMA

SEQ ID NO: 2 - DNA, 1155 bp, Artificial Sequence
  Description of Artificial Sequence Synthetic polynucleotide
atggcgtcagtcattatcgatgacaccacgctgcgtgatggcgaacagtcggctggtgtggcgtttaacgccgatgaaaaaattgctatcgcgcgtgcgctggcagaactgggtgttccggaactggaaattggcatcccgagtatgggtgaagaagaacgtgaagtcatgcatgctattgcgggcctgggtctgagctctcgtctgctggcgtggtgccgcctgtgtgatgtggacctggcggcggcacgctccaccggtgtgacgatggttgatctgtcactgccggtttcggacctgatgctgcatcacaaactgaatcgtgatcgtgactgggcactgcgtgaagttgcacgcctggtcggcgaagcacgtatggctggtctggaagtgtgcctgggctgtgaagatgcgtctcgcgccgacctggaatttgtggttcaggtcggtgaagtggcacaggctgcaggtgctcgtcgcctgcgttttgcggataccgttggtgtcatggaaccgttcggcatgctggatcgttttcgcttcctgagccgtcgcctggacatggaactggaagtgcatgcgcacgatgacttcggtctggcaaccgcaaacacgctggcagcagtgatgggtggtgcaacccatattaacaccacggttaatggcctgggtgaacgtgcaggcaacgctgcgctggaagaatgcgttctggctctgaaaaatctgcacggcattgataccggtatcgacacgcgcggtattccggcaatcagcgctctggtggaacgtgcatctggccgccaggttgcctggcaaaaaagtgtcgtgggcgcgggtgtcttcacccatgaagccggcatccacgtggatggtctgctgaaacatcgtcgcaactatgaaggtctgaatccggatgaactgggccgcagtcactccctggttctgggcaaacatagcggtgcacacatggtccgtaacacgtaccgcgatctgggtattgaactggcagactggcagtctcaagctctgctgggccgtatccgcgcctttagtacccgtacgaaacgttccccgcagccggcagaactgcaagatttctatcgccagctgtgtgaacaaggtaatccggaactggccgcaggcggtatggcctaa

SEQ ID NO: 3 - DNA, 1017 bp, Artificial Sequence
  Description of Artificial Sequence Synthetic polynucleotide
atgcgtctggcggttattgaaggcgatggtatcggccgcgaaattatcccggcggccgttaaagtcctggacgcctttggcctggaatttgaaaaagtgccgctggaactgggctatacccgttgggaacgcaccggtacggcaattagcaacaatgatctggaaacgatcaaaggctgcgacgcggtcctgtttggtgccattaccaccgtgccggacccgaattataaaagcgtgctgctgaccatccgtaaagaactggacctgtacgctaacgtgcgcccggttaaaccgctgccgggtattaccggcgtcacgggtcgtaacgattttgacttcattatcgttcgcgaaaataccgaaggcctgtatagcggtattgaagaaatcggcccggaactgtcttggaccaaacgtgtggttacgcgcaaaggtagcgaacgtattgcggaatacgcctgcaaactggcgaaaaaacgtaaaaacaaactgaccatcgtccataaaagcaatgtgctgaaatctgataaactgtttctggacgtgtgtcgtcagacggcaagtgctaacggcgtggaatatgaagatatgctggttgacagcatggcgtataatctgattatgcgtccggaacgctacgatatcgtcgtgaccacgaacctgttcggtgatattctgtcagacatgtgcgcagctctggttggcagtctgggtctggtcccgtccgcaaatatcggcgaaaaatacgcgtttttcgaaccggtgcacggttccgcaccggatattgctggtaaaggcatcgcgaacccgctggcggccattctgtgtgttaaaatgctgctggaatggatgggcgaaccgcgctcacagattatcgatgaagcgatcgcctatgtgctgcagaagaaaattatcaccccggatctgggcggcaccgcctcgacgatggaagtcggtaacgcagtggctgaatacgttctgtcaaatattcaagatcgtcgctcgccgccgtggtaa

SEQ ID NO: 4 - DNA, 128 bp, Artificial Sequence
  Description of Artificial Sequence Synthetic polynucleotide
aaatcatgaaaaatttatttgctttgtgagcggataacaattataatagcatgctggtcagtattgagcgatgcatgcacggtttccctctagaaataattttgtttaacttttaggaggtaaaaatc

SEQ ID NO: 5 - DNA, 1020 bp, Artificial Sequence
  Description of Artificial Sequence Synthetic polynucleotide
atgcgtaataccccgaaaatctgtgttatcaacggcgacggtatcggcaatgaagttatcccggaaacggtgcgtgtgctgaatgaaattggcgattttgaatttatcgaaacccatgcgggctatgaatgctttaaacgttgtggtgatgcaattccggaaaaaacgattgaaatcgctaaagaaagtgactccatcctgttcggttcagtcaccacgccgaaaccgaccgaactgaaaaacaaaccgtatcgttcgccgattctgacgctgcgcaaagaactggatctgtacgccaatatccgtccgaccttcaacttcaaaaacctggacttcgtgatcatccgcgaaaacacggaaggcctgtacgttaaaaaagaatactacgatgagaaaaacgaagtcgcgaccgccgaacgtattatcagcaaattcggtagctctcgcattgtgaaatttgcgttcgattatgcgctgcaaaacaaccgtaaaaaagtttcttgcatccacaaagcgaacgtcctgcgcatcaccgacggcctgtttctgggtgtgttcgaagaaattagtaaaaaatacgaaaaactgggcattgtttccgatgactatctgatcgatgcaacggctatgtacctgatccgtaacccgcaaatgtttgacgtgatggttaccacgaacctgtttggtgatattctgtcagacgaagcggcgggtctgatcggcggtctgggcatgtcaccgtcggccaacattggcgataaaaatggtctgtttgaaccggttcatggctccgcaccggacattgctggcaaaggtatcagcaatccgattgcaaccatcctgagcgcggcgatgatgctggatcacctgaaaattaacaaagaagcggaatatatccgcaatgccgtgaagaaaaccgtggaatgtaaatacctgacgccggatctgggcggtcatctgaaaaccagcgaagttaccgaaaaaattatcgaaagtattaaaagccaaatgattcagtga

SEQ ID NO: 6 - DNA, 33 bp, Artificial Sequence
  Description of Artificial Sequence Synthetic primer
ccggatcctaccatggcgtcagtcattatcgat

SEQ ID NO: 7 - DNA, 40 bp, Artificial Sequence
  Description of Artificial Sequence Synthetic primer
ctagaagcttcctaaagcaggttaggccataccgcctgcg

(19) United States
(12) Patent Application Publication
(10) Pub. No.: US 2015/0225329 A1
(43) Pub. Date: Aug. 13, 2015

(54) ALTERNATIVE PATHWAYS TO ADIPIC ACID BY COMBINED FERMENTATION AND CATALYTIC METHODS

(71) Applicant: BioAmber Inc., Plymouth, MN (US)

(72) Inventors: James R. Millis, Plymouth, MN (US); Man Kit Lau, Minneapolis, MN (US)

(21) Appl. No.: 14/423,467

(22) PCT Filed: Sep. 11, 2013
(86) PCT No.: PCT/US2013/059170

§ 371 Date: Feb. 24, 2015

Related U.S. Application Data
(60) Provisional application No. 61/701,096, filed on Sep. 14, 2012.

(51) Int. Cl. C07C 51/38; C12P 7/48; C07C 51/36; C07C 51/377; C07C 51/347

(52) U.S. Cl. CPC C07C 51/38; C07C 51/377; C07C 51/347; C07C 51/36; C12P 7/48

(57) ABSTRACT

Processes process for producing adipate or adipic acid using biological pathways and chemical catalyzes are disclosed. Homocitric acid may be a substrate in reaction pathways leading to adipic acid or a salt thereof.

### SEQUENCE LISTING

The instant application contains a Sequence Listing which has been submitted in ASCII format via EFS-Web and is hereby incorporated by reference in its entirety. Said ASCII copy, created on Sep. 10, 2013, is named DNP-10-1205WO_SL.txt and is 9,164 bytes in size.

### TECHNICAL FIELD

This disclosure relates to methods of producing adipates and adipic acid.

### BACKGROUND

Currently, many carbon containing chemicals are derived from petroleum based sources. Reliance on petroleum-derived feedstocks contributes to depletion of petroleum reserves and the harmful environmental impact associated with oil drilling.

Certain carbonaceous products of sugar fermentation are seen as replacements for petroleum-derived materials for use as feedstocks for the manufacture of carbon-containing chemicals. Such products include adipic acid and adipates.

Adipic acid represents a large market for which all commercial production today is petroleum-derived. Adipates such a 3-ketoadipate, 3-hydroxyadipate and hexenedioate are also useful precursors to a wide range of functionalized diacids,

### SUMMARY

We provide a process for producing adipic acid or an adipate including: a) condensing α-ketoglutarate with acetyl-CoA to form homocitrate; b) converting homocitrate to adipate or adipic acid by at least one chemical reaction; and c) optionally, isolating adipate or adipic acid.

We also provide a process of producing adipic acid or an adipate thereof including: a) providing homocitrate; b) decarboxylating homocitrate to form 3-ketoadipate; and c) converting 3-ketoadipate to form adipic acid or adipate directly or through at least one intermediate selected from 3-hydroxyadipate and hexenedioate.

We also provide a process of producing adipic acid esters thereof including: a) providing homocitrate; b) decarboxylating homocitrate to form 3-ketoadipate; c) converting 3-ketoadipate to a 3-ketoadipic acid ester; d) converting 3-ketoadipic acid ester to form an ester of adipic acid directly or through at least one intermediate selected from 3-hydroxyadipic ester and hexenedioate ester; and e) optionally converting the ester of adipic acid to adipic acid.

We further provide a process for producing adipate or an acid thereof comprising: a) providing homocitrate; b) treating the homocitrate to form homocitric acid lactone; c) dehydrogenating homocitric acid lactone to form 4-carboxy-muconolactone; d) decarboxylating 4-carboxy-muconolactone to form 5-carbomethoxy-GBL-4-ene; e) tautomerization of 5-carbomethoxy-GBL-4-ene to form 3-ketoadipate; and f) optionally converting 3-ketoadipate to adipate or adipic acid.

### BRIEF DESCRIPTION OF THE DRAWINGS

FIG. 1 schematically shows a series of pathways that produce adipates and adipic acid from homocitrate.

FIG. 2 schematically shows conversion of homocitrate to adipic acid or adipate through homocitrate lactone.

FIG. 3. is a schematic diagram of plasmid pBA006 constructed to include E. coli codon-optimized homocitrate synthase (nifV) and homoisocitrate dehydrogenase (aksF_Mm) genes.

FIG. 4. is a schematic diagram of plasmid pBA066 constructed to include E. coli codon-optimized homocitrate synthase (nifV), homoisocitrate dehydrogenase (aksF_C5).

FIG. 5 shows the results of homocitrate synthase Activity in BA066 Crude Lysate compared to control cells (BL21).

FIG. 6 is an SDS-PAGE of the insoluble and soluble fraction of cell lysates of BA066 cells transformed with plasmid pBA066 compared to control cells (BL21).

### DETAILED DESCRIPTION

Combined biological and thermochemical routes to industrial chemicals, can often be a faster and more economical route compared with multi-step biochemical pathways. Such pathways often provide valuable intermediates that also have commercial value. This approach may be applied to the production of adipic acid, adipates and esters of adipic acid. For example, we provide a number of chemical and biochemical pathways that utilize homocitrate and 3-ketoadipate as starting compounds and/or chemical intermediates.

The disclosed biochemical pathways may include the activity of one or more proteins or enzymes, particularly heterologous enzymes, that catalyze reactions converting a substrate to a product or intermediate in a pathway. Microorganisms may be modified to express one or more of the proteins or enzymes by techniques well known in the art. Accordingly, we provide engineered metabolic routes, isolated nucleic acids or engineered nucleic acids, polypeptides or engineered polypeptides, host cells or genetically engineered host cells, methods and materials to produce compounds and intermediates of interest from a carbon source.

Carbon sources suitable as a starting point of our biosynthetic pathways include carbohydrates and synthetic intermediates. Examples of carbohydrates which cells are capable of metabolizing include sugars, such as glucose, dextroses, triglycerides and fatty acids. Intermediate products from metabolic pathways, such as 2-ketoglutatrate can also be used as starting points.

Those skilled in the art will understand that engineered pathways exemplified herein are described in relation to, but are not limited to, species specific genes and encompass homologs or orthologs of nucleic acid or amino acid sequences. Homologous and orthologous sequences possess a relatively high degree of sequence identity/similarity when aligned using methods known in the art.

Aspects of our methods and microorganisms relate to “genetically modified” or recombinant microorganisms or host cells that have been engineered to possess new metabolic capabilities or new metabolic pathways. As used herein the term “genetically modified” microorganisms includes microorganisms having at least one genetic alteration not normally found in the wild type strain of the referenced species such as expression of a recombinant gene. In some examples, genetically engineered microorganisms are engineered to express or overexpress at least one particular enzyme at critical points in a metabolic pathway, and/or suppress or block the activity of other enzymes, to overcome or circumvent metabolic bottlenecks.

We provide genetically modified host cells or microorganisms and methods of using the same to produce adipic acids and adipates from alpha-keto acids. A “host cell” as used herein refers to a eukaryotic cell, a prokaryotic cell or a cell from a multicellular organism (e.g. cell line) cultured as a unicellular entity. A host cell may be prokaryotic (e.g., bacterial such as E. coli or B. subtilis) or eukaryotic (e.g., a yeast, mammal or insect cell). For example, host cells may be bacterial cells (e.g., Escherichia coli, Bacillus subtilis, Mycobacterium spp., M. tuberculosis, or other suitable bacterial cells), Archaea (for example, Methanococcus Jannaschii or Methanococcus Maripaludis or other suitable archaic cells), yeast cells (for example, Saccharomyces species such as S. cerevisiae, S. pombe, Picchia species, Candida species such as C. albicans, or other suitable yeast species). Preferred host cells include E. coli.

The metabolically engineered cell may be made by transforming a host cell with at least one nucleotide sequence encoding an enzyme involved in the engineered metabolic pathways. As used herein the term “nucleotide sequence”, “nucleic acid sequence” and “genetic construct” are used interchangeably and mean a polymer of RNA or DNA, single- or double-stranded, optionally containing synthetic, non-natural or altered nucleotide bases. A nucleotide sequence may comprise one or more segments of cDNA, genomic DNA, synthetic DNA, or RNA.

In a preferred example, the nucleotide sequence encoding enzymes or proteins in a metabolic pathway is codon-optimized to reflect the typical codon usage of the host cell without altering the polypeptide encoded by the nucleotide sequence. In selected examples, the term “codon optimization” or “codon-optimized” refers to modifying the codon content of a nucleic acid sequence without modifying the sequence of the polypeptide encoded by the nucleic acid to enhance expression in a particular host cell. In selected examples, the term is meant to encompass modifying the codon content of a nucleic acid sequence as a mean to control the level of expression of a polypeptide (e.g. either increase or decrease the level of expression).

In some examples, a metabolically engineered cell may express one or more polypeptide having an enzymatic activity necessary to perform the steps described below. For example, a particular cell may comprise one, two, three, four, five or more than five nucleic acid sequences, each one encoding the polypeptide(s) necessary to perform the conversion of a substrate to a product in the pathway, such as pathway converting alpha-ketoglutarate or homocitrate to adipic acid or adipate. Alternatively, a single nucleic acid molecule can encode one, or more than one, polypeptide. For example, a single nucleic acid molecule can contain nucleic acid sequences that encode two, three, four or more different polypeptides.

Nucleic acid sequences useful for the methods and microorganisms described herein may be obtained from a variety of sources such as, for example, amplification of cDNA sequences, DNA libraries, de novo synthesis, and/or excision of one or more genomic segments. The sequences obtained from such sources may then be modified using standard molecular biology and/or recombinant DNA technology to produce nucleic sequences having desired modifications. Exemplary methods for modification of nucleic acid sequences include, for example, site directed mutagenesis, PCR mutagenesis, deletion, insertion, substitution, swapping portions of the sequence using restriction enzymes, optionally in combination with ligation, homologous recombination, site specific recombination or various combination thereof. In other examples, the nucleic acid sequences may be a synthetic nucleic acid sequence. Synthetic polynucleotide sequences may be produce using a variety of methods described in U.S. Pat. No. 7,323,320, the subject matter of which is incorporated herein by reference in its entirety.

Methods of transformation for bacteria, plant, and animal cells are known. Common bacterial transformation methods include electroporation and chemical modification.

To take advantage of chemical pathways, chemical products may be isolated and treated accordingly to techniques known in the art.

It is well recognized in the art that adipates can be readily converted to adipic acids and, conversely, adipic acids can be readily converted to adipates. Accordingly, it should be appreciated that the term “adipate(s)” may be used interchangeably with “adipic acid(s)” where one can readily be converted to or substituted for the other. Similarly, other compounds having acid and salt forms referred to herein may be referred to by their acid or salt forms interchangeably. Thus, for example, one skilled the art would understand that a reaction pathway described as forming the acid form of a compound as an intermediate or product may also be used to form the salt form of the compound.

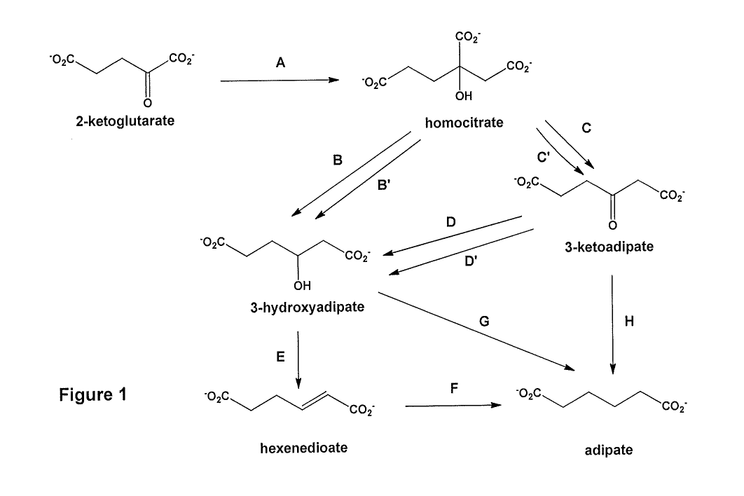

FIG. 1 shows an exemplary biological and/or chemical pathway for the biosynthesis of adipic acid and adipates from 2-ketoglutarate. Homocitrate (Step A in FIG. 1) may be readily prepared using biological techniques. Homocitrate synthase enzymes (EC 2.3.3.14) catalyze the chemical reaction acetyl-CoA+H₂O+2-oxoglutarate⇄homocitrate+CoA. The product, homocitrate, is also known as (R)-2-hydroxybutane-1,2,4-tricarboxylate.

For example, a homocitrate synthase askA may be derived from Methanococcus jannaschii. Methanococcus jannaschii is a thermophilic methanogen and the coenzyme B pathway in this organism has been characterized at 50-60° C. Accordingly, enzymes originating from Methanococcus jannaschii, such as homocitrate synthase askA, may have peak efficiency at higher temperatures around about 50-60° C. However, alternative AksA protein homologs from other methanogens that propagate at a lower temperature may also be used.

In some preferred examples, synthesis of homocitrate may be catalyzed by the homocitrate synthase NifV or NifV homologs. Homologs of NifV are found in a variety of organisms including, but not limited to, Azotobacter vinelandii, Klebsiella pneumoniae, Azotobacter chroococcum, Frankia sp. (strain FaCl), Anabaena sp. (strain PCC 7120), Azospirillum brasilense, Clostridium pasteurianum, Rhodobacter sphaeroides, Rhodobacter capsulatus, Frankia alni, Carboxydothermus hydrogenoformans (strain Z-2901/DSM 6008), Anabaena sp. (strain PCC 7120), Frankia alni, Enterobacter agglomerans, Erwinia carotovora subsp. atroseptica (Pectobacterium atrosepticum), Chlorobium tepidum, Azoarcus sp. (strain BH72), Magnetospirillum gryphiswaldense, Bradyrhizobium sp. (strain ORS278), Bradyrhizobiuni sp. (strain BTAi1/ATCC BAA-1182), Clostridium kluyveri (strain ATCC 8527/DSM 555/NCIMB 10680), Clostridium kluyveri (strain ATCC 8527/DSM 555/NCIMB 10680), Clostridium butyricum 5521, Cupriavidus taiwanensis (strain R1/LMG 19424), Ralstonia taiwanensis (strain LMG 19424), Clostridium botulinum (strain Eklund 17B/type B), Clostridium botulinum (strain Alaska E43/type E3), Synechococcus sp. (strain JA-2-3B′a(2-13)) (Cyanobacteria bacterium Yellowstone B-Prime), Synechococcus sp. (strain JA-3-3Ab) (Cyanobacteria bacterium Yellowstone A-Prime), Geobacter sulfurreducens and Zyniomonas mobilis. In preferred examples, homocitrate synthase is NifV from Azotobacter vinelandii and may have an amino acid sequence according to SEQ ID NO: 1.

In other preferred examples, homocitrate synthase is NifV from Azotobacter vinelandii and is encoded by a nucleotide sequence according to SEQ ID NO: 2, which is codon-optimized for expression in E. coli. In other examples, the first step of the pathway may be engineered to be catalyzed by the homocitrate synthase Lys 20 or Lys 21. Lys 20 and Lys 21 are two homocitrate synthase isoenzymes implicated in the first step of the lysine biosynthetic pathway in the yeast Saccharomyces cerevisiae. Homologs of Lys 20 or Lys 21 are found in a variety of organisms such as Pichia stipitis and Therms thermophilus.

In some examples, enzymes catalyzing the reaction involving acetyl coenzyme A and alpha-keto acids as substrates are used to convert alpha-ketoglutarate into homocitrate (e.g. EC 2.3.3.-) may originate from Methanogenic archaea. Methanogenic archaea contain three closely related homologs of AksA: 2-isopropylmalate synthase (LeuA) and citramalate (2-methylmalate) synthase (CimA) which condenses acetyl-CoA with pyruvate. This enzyme is believed to be involved in the biosynthesis of isoleucine in methanogens and possibly other species lacking threonine dehydratase. In some examples, the acyl transferase enzyme is an isopromylate synthase (e.g. LeuA, EC 2.3.3.13) or a citramalate synthase (e.g. CimA, EC 2.3.1.182). The cellular intermediate, homocitrate, may then be converted to adipate or adipic acid by several routes as shown in FIG. 1.

As shown in FIG. 1, homocitrate may be biologically converted into 3-hydroxyadipate (Step B) or 3-ketoadipate (Step C) using different types of decarboxylases. A decarboxylase removes a carbon dioxide from the target substrate. In nature, decarboxylation of homocitrate follows a series of reactions. Homocitrate is first dehydrated into cis-homoaconitate. Rehydration of cis-homoaconitate produces threo-iso-homocitrate. The C3 hydroxy group shifted to C2 position after these hydration/dehydration reactions. Finally, decarboxylation of threo-iso-homocitrate produces 2-ketoadipate as final product.

However, as shown in FIG. 1, Step B, homocitrate may be converted into 3-hydroxyadipate by decarboxylases that are active toward eliminating CO₂ from an α-hydroxycarboxylate functionality are of particular interest for catalyzing the reaction converting homocitrate to 3-hydroxyadipate. For example, α-acetolactate decarboxylase (EC 4.1.1.5) natively decarboxylates acetolactate to produce acetoin (Goupil-Feuillerat, N.; Cocaign-Bousquet, M.; Godon, J-J.; Ehrlich, S. D.; Renault, P. J. Bacteriol. 1997, 179, 6285). It had been reported that α-acetolactate decarboxylase from Aerobacter aerogenes is capable of catalyzing a reaction using a non-native 2-hydroxy-2-ethyl-3-oxobutanoate as substrate (Stormer, F. C. Methods Enzymol. 1975, 41B, 518). Arylmalonate decarboxylase (EC 4.1.1.76) had been reported to catalyze the conversion of α-arylmalonates into α-arylcarboxylic acids. Arylmalonate decarboxylase is highly robust and does not require cofactors to increase the potential of this enzyme for biocatalysis (Miyamoto, K.; Ohta, H. Eur. J. Biochem. 1992, 210, 475). More recently, structure-guided directed evolution has been employed to alter the specificities of this enzyme (Okrasa, K.; Levy, C.; Wilding, M.; Godall, M.; Baudendistel, N.; Hauer, B.; Leys, D.; Micklefield, J. Angew. Chem. Int. Ed. 2009, 48, 7691).

As shown in FIG. 1, Step C, homocitrate may be converted into 3-ketoadipate following an oxidative decarboxylation mechanism. Besides releasing carbon dioxide, this particular type of decarboxylase may simultaneously oxidize the α-hydroxy into an oxo functionality. Such an enzyme was found in the fatty acid degradation pathway. For example, α-hydroxy acid decarboxylase from brain microsomes had been reported to catalyze the decarboxylation of α-hydroxystearic acid (Levis, G. M.; Mead, J. F. J. Biol. Chem. 1964, 239, 77). As another example, CloR encoding non-heme iron oxygenase had been reported to catalyze two consecutive oxidative decarboxylations within a single biosynthetic pathway of clorobiocin (Pojer, F.; Kahlich, R.; Kammerer, B.; Li, S. M.; Heide, L. J. Biol. Chem. 2003, 278, 30661). CloR activity had been recently studied by a functional model, suggesting that the oxidative decarboxylation of mandelate occurred upon exposure to oxygen (Paine, T. K.; Paria, S.; Que Jr., L. Chem, Commun. 2010, 46, 1830).

Alternatively, the oxidative decarboxylation of homocitrate to 3-ketoadipate may also be done using a spontaneous biological process. In this pathway, the first step is the enzymatic oxidation of the C-3 hydroxyl to form the keto form of the tricarboxylate, believed to be an unstable intermediate that will spontaneously decarboxylate to 3-keto adipate. Representative enzymes that catalyze this reaction include dehydrogenases, such as malate dehydrogenase (EC 1.1.1.37) or similar oxidoreductases. Cofactors for this reaction can include NAD or NADP.

Alternately, as shown in FIG. 1, it is possible to use a chemical catalyst to perform decarboxylation of homocitrate to 3-hydroxyadipate (Step B′). Common chemical catalysts, such as Bronsted or Lewis acids, will facilitate this reaction. (J. Mol. Evolution (1972) V1(4), pp 326 and J. Org. Chem. (1989) V54(18) 6310). Typical Lewis acids include salts of aluminum, lanthanum, iron and cerium. Solid Lewis acids such as alumina, silica-alumina, niobia hydrate and sulfonated zirconia may also be used. Oxidative decarboxylation may also be used to produce 3-hydroxyadipate. Suitable oxidants such as hydrogen peroxide, peroxy mono-sulfate and oxygen may be used in the presence of homogeneous catalysts such as porphyrin or EDTA complexes of vanadium, cobalt, manganese, iron and copper.

Photochemical decarboxylation of homocitrate or homocitric acid (B′) to 3-hydroxyadipate or 3-hydroxyadipic acid may also be used. This can be done through the action of light in the presence of a photo catalyst such, as TiO₂, or various multivalent metal titanates. (U.S. Pat. No. 4,515,667, and U.S. Pat. No. 4,303,486). Typically, an aqueous solution of homocitrate (5%-50% by weight preferably 5% to 40%, or any amount therebetween) is contacted with an appropriate amount of TiO₂ catalyst and stirred well while maintaining a temperature of 0° C.-100° C., preferably 20° C.-30° C., for 30 minutes to 24 hrs, preferably 15-24 hrs, while also being exposed to incident light energy with wavelength of between 2000A and 15,000A (ultraviolet to infrared), preferably 2000A to 5000 A. The amount of solid titanium-based catalyst in the slurry can be in the range of 2 to 100 mgs catalyst/ml of homocitrate solution and is preferably in the range of 5 to 50 mgs/ml of homocitrate solution. The gas atmosphere covering the slurry of catalyst and homocitrate solution can be air, oxygen or an inert gas, such as nitrogen, helium or argon, and the pressure may be 1-10 atmospheres, preferably 1-3 atmospheres. In addition to TiO₂, the catalyst may comprise Ba, Mn, Fe, Sr, Ca, Mg, Zn or Bi titanates and may be a granular or powder form. The catalyst may be used in its pure oxide form or modified by the incorporation of a metallic catalyst comprising or consisting of platinum. The incorporation of Pt may be done via any method known to those skilled in the art of making platinum catalysts.

Still referring to FIG. 1, oxidative decarboxylation of homocitrate to 3-ketoadipate (Step C′) using chemical catalysis can be assisted by homogeneous catalysts, for example those composed of manganese or iron complexes Chinese J. Chem., (2009), V27(5), 1007, and ARKIVOC, (2008), V11, 238 and Egyptian J. of Chem., (1973) 131-7). Alternately copper or cobalt containing catalysts may be employed (EP 518441 and Fette, Seifen Anstrichmittel, (1973) V75(6), 388 and Tetrahedron, (2001), V57(6), 1075). Various oxidants may be employed such as air, oxygen, periodates, persulfates, per-borates, hydrogen peroxide and mono-oxypersulfates. Temperatures in the range of 60° C.-400° C. and pressures from atmospheric to 250 atmospheres may be employed. Suitable solvents include, but are not limited to, hydrocarbons, water and glycol ethers. Alternately solid heterogeneous catalysts may be employed for oxidative decarboxylation with air or oxygen. These solid catalysts may be composed of various oxides such as tin oxide, bismuth oxide, zinc oxide, molybdenum and tungsten oxides and the like. These oxides may be used alone or in combinations and also with the optional incorporation of basic oxides such as potassium, sodium, cesium, magnesium, strontium, barium and calcium oxide (J. Catal., (1977) V50, 291 and J. of Ind. & Engineering Chem. (2011), v17(4), 788).

Decarboxylation of homocitrate (C′) can also be effected using purely thermal means without a catalyst in the temperature range of 200° C. to 500° C. and residence times at temperature of from 10 minutes to 300 minutes (J. Anal. Appl. Pyrolysis 71 (2004) 987-996 and J. Am. Oil Chem. Soc. 65 (1988) 1781, J. Agr. Food Chem. 31 (1983) 1268, J. Anal. Appl. Pyrolysis 29 (1994) 153, J. Braz. Chem. Soc. 10 (1999) 469, and Energy Fuels 10 (1996) 1150, and Ind. Eng. Chem. Res., (2008), V47(15), 5328). Preferably no solvent is employed for thermal decarboxylation, but suitable solvents, including hydrocarbons, water and glycol ethers, may be used. Inert gas atmospheres or air may be employed with inert gases such as argon, nitrogen or helium preferred.

Catalytic decarboxylation of homocitrate to 3-ketoadipate (Step C′) may also be effected by various catalysts such as those comprising palladium, platinum, silver, nickel, cobalt or iridium on solid supports such as carbon, alumina, silica-alumina, zirconia, titania, tungsten oxide and niobium oxide and combinations of these (Ind. Eng. Chem. Res., (2006), V45, 5708, Fuel, (2008), V87 933-945, Fuel, (2012), V95, 622, ChemSusChem, (2009), V2, 581, Hydrocarbons for diesel fuel via decarboxylation of vegetable oils, 2005; pp 197, Chemische Berichte-Recueil 1982, 115, (2), 808, Energy & Fuels 2007, 21, (1), 30-41, Fuel 2008, 87, (17-18), 3543, Chemical Industries (Boca Raton, Fla., United States) 2007, 115, (Catalysis of Organic Reactions), 415, Applied Catalysis, A: General (2009), 355, (1-2), 100, Topics in Catalysis, (2011), V54(8-9), 460, U.S. Pat. No. 4,554,397A, (1985), and U.S. Pat. No. 3,476,803 (1968)). The metallic component of the catalyst may be employed at levels of between 0.1% to 10% by weight and the preferred temperatures are in the range of 250° C. to 450° C. See, Goosen, et.al., in Pure and Applied Chemistry (2008) V80(8) 1725-33. Alternately zeolites or other solid acids may be employed for catalytic decarboxylation at temperatures of 300° C.-500° C. and short residence time with or without added metallic components (GB2039943A, (1979), WO 2007 136873A3 and US 2007/0281875 and Energy & Fuels, (2008), V22(3), 1923).

Still referring to FIG. 1, the biological reduction of 3-ketoadipate to 3-hydroxyadipate (Step D) or to adipate (Step H), can be performed using oxidoreductases. The oxidation-reduction sequence of these two steps allows for efficient cofactor recycle.

As shown in FIG. 1, 3-ketoadipate may be converted to 3-hydroxyadipate (Step D) by using a dehydrogenase. In some cases, the oxidizing equivalent can be supplied in the form of an NAD+ or NADP+. Preferably, such dehydrogenase can be one that uses secondary alcohols as substrates. In addition to the malate dehydrogenase, such dehydrogenase can be E. coli AdhP or AdhE that are known to have broad substrate specificities. S. cerevisiae and S. carlsbergensis ADH1 was reported to convert 2-butanol to butanone (Pal, S.; Park, D. H.; Plapp, B. V. Chem. Biol. Interact. 2009, 178, 16). A thermostable alcohol dehydrogenase from Thermus sp. ATN1 had been reported to use 1-phenyl-2-propanol and cyclohexanol as substrates to produce its corresponding ketones (Hoellrigl, V.; Hollann, F.; Kleeb, A. C.; Buehler, K.; Schmid, A. Appl. Microbiol. Biotechnol. 2008, 81, 263). 3-hydroxyacyl-CoA dehydrogenase (EC 1.1.1.35), for example, E. coli FadJ and FadB, are suitable NAD-dependent dehydrogenases. They had been reported to catalyze the conversion of (S)-3-hydroxybutyryl-CoA into acetoacetyl-CoA (Binstock, J. F.; Schulz, H. Methods Enzymol. 1981, 71, 403.) On the other hand, acetoacetyl-CoA reductase (EC 1.1.1.36) from Azotobacter beijerinckii is a suitable NADP-dependent dehydrogenase. It has been reported to catalyze the reverse reaction and produce the reduced hydroxyl compound for PHB synthesis. It has been also reported that 3-hydroxypimeloyl-CoA could be reduced to 3-oxopimeloyl-CoA in Rhodopseudomonas palustris during the benzene ring degradation (Harwood, C. S.; Gibson, J. J. Bacteriol. 1997, 179, 301. This reaction is of particular interest due to the structurally similar properties between 3-ketoadipate and 3-oxopimeloyl-CoA.

Still referring to FIG. 1, the reduction of 3-ketoadipate to 3-hydroxyadipate (Step D′) can also be accomplished with suitable metal catalysts. Catalysts for keto acid reductions to hydroxyl acids include, but are not limited to, hetero and homogeneous ruthenium examples and also homogeneous rhodium examples. Ruthenium is a preferred metal, although supported platinum and palladium catalysts as well as copper and nickel, including alkaloid modified RANEY® nickel have been used. Carbon is also a suitable support, but alumina and calcium carbonate may also be used (The Catalytic Reaction Guide (2007) Johnson Matthey Catalysts, U.S. Pat. No. 4,933,482, U.S. Pat. No. 5,387,696, React. Kim. Catal. Letters (1975), V2, 257, Inorg. Chem. Acta, (1977) V25, L61, Nanoparticles and Catalysis, Didier Astruc, ed. Wiley-Verlag (2008) Weinheimm Ger., p 373, JACS, (2008) V130(44) 14483, AICHE 2011 Annual Meeting paper #247f Oct., 18, 2011, JACS, (1939), v61(4), 843, Stud Surf, Sci & Catal., (1993), V78, 139, Chemistry, (2007), V13(32), 9076, and the review in Catalysis by Metal Complexes (2006), V31, 77-160). Other examples include non-catalytic transfer hydrogenations with formic acid as the hydrogen donor (AIP Conference Proceedings, Nov. 25, 2010, Vol 1251 (1) p 356). Generally mild temperatures in the range of 75° C. to 150° C. and moderate hydrogen pressures are shown effective in the range of about 20 psig to around 1000 psig. Water is a preferred solvent but methanol, ethanol or isopropanol, tetrahydrofuran, dioxane, acetic acid and mixtures of these and others are also acceptable.

As shown in FIG. 1, the catalytic reduction of 3-ketoadipate or 3-ketoadipic acid to adipate or adipic acid (Step H in FIG. 1) may be accomplished by homogeneous or heterogeneous hydrogenation catalysts. Suitable catalysts include supported Group VIII metals and Raney catalysts described below. Keto compounds can be hydrogenolyzed to the corresponding hydrocarbon (U.S. Pat. No. 4,067,900) by use of a homogeneous Ir or Rh complex composed of generally [M(CO)aX4-a]-c where M=It or Rh, a is 1-3, and c is 1 or 2. The preferred conditions are 100-240° C., 10-1000 psig, 150-200° C. and almost any Ir or Rh material capable of being converted to the complex can be the Ir or Rh precursor. Additionally, I or Br may be added in form of LH or LiBr and/or HBr or HI. The preferred solvents include, but are not limited to, simple or halogenated hydrocarbons or aromatics, or acids. Acetic or propionic acids are preferred solvents.

As shown in FIG. 1, 3-hydroxyadipate may be accumulated by any of the above methods and dehydrated biologically to hexenedioate (Step E) by using a dehydratase or hydro-lyase. A dehydratase or hydrolyase catalyzes a double-bond forming reaction by the elimination of a water molecule. Enzymes that catalyze substrates structurally similar to 3-hydroxyadipate may be used in this proposed transformation.

For example, E. coli fumarases (EC 4.2.1.2) FumA, FumB and FumC had been reported to catalyze the formation of fumarate from malate (Tseng, C. P.; Yu, C. C.; Lin, H. H.; Chang, C. Y.; Kuo, J. T. J. Bacteriol. 2001, 183, 461). The dimethylmaleate hydratase (EC 4.2.1.85) from Eubacterium barkeri is also suitable and had been reported to catalyze the hydration reaction using substituted malate as substrate. This enzyme catalyzes the formation of 2,3-dimethylmalate from dimethylmaleate. E. coli aconitate hydratase (EC 4.2.1.3) catalyzes the conversion of citrate into cis-aconitate and may also be used (Tsuchiya, D.; Shimizu, N.; Tomita, M. Biochim. Biophys. Acta 2008, 1784, 1847). The sequence and expression of the E. coli carnitine dehydratase (EC 4.2.1.89) had been reported and this enzyme catalyzes the formation of carnitine from crotonobetaine (Eichler, K.; Schunck, W. H.; Kleber, H. P.; Mandrand-Berthelot, M. A. J. Bacteriol. 1994, 176, 2970). Carnitine dehydratase may also be used to catalyze Step E.

Alternatively, this dehydration reaction may also proceed through its CoA ester or acyl-carrier-protein (ACP) derivatives, 3-hydroxyadipyl-CoA or 3-hydroxyadipyl-ACP, respectively. For examples, 2-Enoyl-CoA hydratase (EC 4.2.1.17) from Pseudomonas putida (PhaJ) and Rattus norvegicus had been reported to catalyze the reaction of 3-hydroxyacyl-CoA into forming 2-enoyl-CoA (Vo, M. T.; Lee, K. W.; Jung, Y. M.; Lee, Y. H. J. Biosci. Bioeng. 2008, 106, 95; Hiltunen, J. K; Palosaari, P. M.; Kunau, W. H. J. Biol. Chem. 1989, 264, 13536). E. coli Crotonyl-ACP hydratase (EC 4.2.1.58) had been reported to catalyze the formation of crotonyl-ACP from 3-hydroxybutanoyl-ACP and may be used (Majerus, P. W.; Alberts, A. W.; Vagelos, P. R. J. Biol. Chem. 1965, 240, 618). Intermediate or long-chain beta-hydroxyacyl-ACP dehydratase (EC 4.2.1.59) in E. coli had also been reported to dehydrate variable chain length of 3-hydroxyacyl-ACP into its corresponding 2-enoyl-ACP products and may also be used (Mizugaki, M.; Swindell, A. C.; Wakil, S. J. Biochem. Biophys. Res. Commun. 1968, 33, 520).

The chemical dehydration of 3-hydroxyadipate to hexendioate (Step E in FIG. 1) may be readily accomplished by the use of homogeneous or heterogeneous acid catalysts (Tetrahedron Letters, (2002) 58(42) 8565, Tetrahedron Letters, (1998) 39(20) 3327 and Ind. Engr. Chem. Res., (2012) 51(18) 6310). Suitable acid catalysts for the present methods are heterogeneous (or solid) acid catalysts. The at least one solid acid catalyst may be supported on at least one catalyst support (herein referred to as a “supported acid catalyst”). Solid acid catalysts include, but are not limited to, (1) heterogeneous heteropolyacids (HPAs) and their salts, (2) natural clay minerals, such as those containing alumina or silica (including zeolites), (3) cation exchange resins, (4) metal oxides, (5) mixed metal oxides, (6) metal salts such as metal sulfides, metal sulfates, metal sulfonates, metal nitrates, metal phosphates, metal phosphonates, metal molybdates, metal tungstates, metal borates, and (7) combinations of groups 1 to 6. When present, the metal components of groups 4 to 6 may be selected from elements from Groups I, IIa, IIIa, VIIa, VIIIa, Ib and IIb of the Periodic Table of the Elements, as well as aluminum, chromium, tin, titanium and zirconium.

Suitable HPAs include compounds of the general Formula Xa MbOcq-, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Non-limiting examples of salts of HPAs are lithium, sodium, potassium, cesium, magnesium, barium, copper, gold and gallium, and onium salts such as ammonia. Methods for preparing HPAs are well known in the art and are described, for example, in Hutchings, G. and Vedrine, J., supra; selected HPAs are also available commercially, for example, through Sigma-Aldrich Corp. (St. Louis, Mo.). Examples of HPAs suitable for the process of this disclosure include tungstosilicic acid (H₄[SiW₁₂O₄₀].xH₂O), tungstophosphoric acid (H₃[PW₁₂O₄₀].xH₂O), molybdophosphoric acid (H3 [PMo₁₂O₄₀].xH₂O), molybdosilicic acid (H₄[SiMo₁₂O₄₀].xH₂O), vanadotungstosilicic acid (H₄₊ₙ[SiVₙW₁₂₋ₙO₄₀].xH₂O), vanadotungstophosphoric acid (H₃₊ₙ[PVnW₁₂₋ₙO₄₀].xH₂O), vanadomolybdophosphoric acid (H₃₊ₙ[PVₙMo₁₂₋ₙO₄₀].xH₂O), vanadomolybdosilicic acid (H₄+n[SiVₙMo₁₂₋ₙO₄₀].xH₂O), molybdotungstosilicic acid (H₄[SiMoₙW₁₂₋ₙO₄₀].xH₂O), molybdotungstophosphoric acid (H₃[PMoₙW₁₂₋₁O₄₀].xH₂O), wherein n in the Formulas is an integer of 1 to 11 and x is an integer of 1 or more.

Natural clay minerals are well known in the art and include, without limitation, kaolinite, bentonite, attapulgite, montmorillonite and zeolites. They may be used in their natural form or after treatment with aqueous acids such as sulfuric acid.

Suitable cation exchange resins for use as solid acid catalyst include, but are not limited to, styrene-divinylbenzene copolymer-based strong cation exchange resins such as AMBERLYST® (Dow; Philadelphia, Pa.), DOWEX® (for example, DOWEX® Monosphere M-31) (Dow; Midland, Mich.), CG resins from Resintech, Inc. (West Berlin, N.J.), and Lewatit resins such as MonoPlus S 100 H from Sybron Chemicals Inc. (Birmingham, N.J.).

Fluorinated sulfonic acid polymers can also be used as solid acid catalysts for the process of the present disclosure. These acids are partially or totally fluorinated hydrocarbon polymers containing pendant sulfonic acid groups, which may be partially or totally converted to the salt form. One particularly suitable fluorinated sulfonic acid polymer is NAFION® perfluorinated sulfonic acid polymer, (E.I. du Pont de Nemours and Company, Wilmington, Del.). One preferred form is NAFION Super Acid Catalyst, a bead-form strongly acidic resin which is a copolymer of tetrafluoroethylene and perfluoro-3,6-dioxa-4-methyl-7-octene sulfonyl fluoride, converted to either the proton (H+), or the metal salt form. NAFION® may also be employed in a supported form, for example supported on silica such as SAC®-13 (BASF).

Preferred solid acid catalysts include cation exchange resins, such as AMBERLYST® 15 (Dow, Philadelphia, Pa.), AMBERLITE® 120 (Dow), NAFION®, and natural clay materials, including zeolites such as mordenite.

When used, the at least one support for the at least one solid acid catalyst can be any solid substance that is inert under the reaction conditions including, but not limited to, oxides such as silica, alumina and titania, compounds thereof or combinations thereof; barium sulfate; zirconia; carbons, particularly acid washed carbon; and combinations thereof. Acid washed carbon is a carbon that has been washed with an acid, such as nitric acid, sulfuric acid or acetic acid, to remove impurities. The support can be in the form of powder, granules, pellets, or the like. The supported acid catalyst can be prepared by depositing the acid catalyst on the support by any number of methods well known to those skilled in the art of catalysis, such as spraying, soaking or physical mixing, followed by drying, calcination, and if necessary, activation through methods such as reduction or oxidation. The preferred loading of the at least one acid catalyst on the at least one support is from about 0.1 weight percent to about 20 weight percent based on the combined weights of the at least one acid catalyst plus the at least one support.

Examples of supported acid catalysts include, but are not limited to, phosphoric acid on silica, NAFION® on silica, HPAs on silica, titania sulfated or tungstated zirconia and sulfated titania.

Hydrogenation of hexenedioate to adipate (Step F) may be readily performed under relatively mild conditions using a variety of catalysts (“The Catalytic Reaction Guide” Johnson Matthey Catalysts (2007) and Chapter 7 in “Fundamentals of Industrial Catalytic Processes” CH Bartholomew and RJ Farrauto, 2nd ed, Wiley-Interscience, (2006) pp 487-559 and R L Augustine, “Heterogeneous Catalysis for the Synthetic Chemist” (1996) Marcel Dekker, NY and PN Rylander “Catalytic Hydrogenation over Platinum Metals”, (1967) Academic Press, NY). A principal component of the catalyst useful for hydrogenation may be selected from metals from the group consisting of palladium, ruthenium, rhenium, rhodium, iridium, platinum, nickel, cobalt, copper, iron, compounds thereof, and combinations thereof. Similar processes described for the hydrogenation of hexenedioate to adipate (Step F), described below, may be used and/or modified to chemically catalyze the conversion of 3-hydroxy adipate to hexenedioate (Step E), 3-hydroxy adipate to adipate (Step G), or 3-ketoadipate to adipate (Step G).

A chemical promoter may be used to augment the activity of the catalyst. The promoter may be incorporated into the catalyst during any step in the chemical processing of the catalyst constituent. The chemical promoter generally enhances the physical or chemical function of the catalyst agent, but can also be added to retard undesirable side reactions. Suitable promoters include metals selected from tin, zinc, copper, gold, silver, and combinations thereof. The preferred metal promoter is tin. Other promoters that can be used are elements selected from Group I and Group II of the Periodic Table.

The catalyst may be supported or unsupported. A supported catalyst is one in which the active catalyst agent is deposited on a support material by a number of methods such as spraying, soaking or physical mixing, followed by drying, calcination and, if necessary, activation through methods such as reduction or oxidation. Materials frequently used as a support are porous solids with high total surface areas (external and internal) which can provide high concentrations of active sites per unit weight of catalyst. The catalyst support may enhance the function of the catalyst agent. The catalyst support can be any solid, inert substance including, but not limited to, oxides such as silica, alumina and titania; barium sulfate; calcium carbonate; and carbons. The catalyst support can be in the form of powder, granules, pellets or the like.

A preferred support material may be selected from the group consisting of carbon, alumina, silica, silica-alumina, silica-titania, titania, titania-alumina, barium sulfate, calcium carbonate, strontium carbonate, compounds thereof and combinations thereof. Supported metal catalysts can also have supporting materials made from one or more compounds. More preferred supports are carbon, titania and alumina. Further preferred supports are carbons with a surface area greater than about 100 m²/g. A further preferred support is carbon with a surface area greater than about 200 m²/g. Preferably, the carbon has an ash content that is less than about 5% by weight of the catalyst support. The ash content is the inorganic residue (expressed as a percentage of the original weight of the carbon) which remains after incineration of the carbon.

A preferred content of the metal catalyst in the supported catalyst may be from about 0.1% to about 20% of the supported catalyst based on metal catalyst weight plus the support weight, or any amount therebetween. A more preferred metal catalyst content range is from about 1% to about 10% of the supported catalyst, or any amount therebetween.

Combinations of metal catalyst and support system may include any one of the metals referred to herein with any of the supports referred to herein. Preferred combinations of metal catalyst and support include palladium on carbon, palladium on alumina, palladium on titania, platinum on carbon, platinum on alumina, platinum on silica, iridium on silica, iridium on carbon, iridium on alumina, rhodium on carbon, rhodium on silica, rhodium on alumina, nickel on carbon, nickel on alumina, nickel on silica, rhenium on carbon, rhenium on silica, rhenium on alumina, ruthenium on carbon, ruthenium on alumina and ruthenium on silica.

Further preferred combinations of metal catalyst and support include ruthenium on carbon, ruthenium on alumina, palladium on carbon, palladium on alumina, palladium on titania, platinum on carbon, platinum on alumina, rhodium on carbon, and rhodium on alumina.

A more preferred support is carbon. Further preferred supports are those, particularly carbon, that have a BET surface area less than about 2,000 m²/g. Further preferred supports are those, particularly carbon, that have a surface area of about 300 to 1,000 m²/g, or any amount therebetween.

A catalyst that is not supported on a catalyst support material is an unsupported catalyst. An unsupported catalyst may be platinum black or a RANEY® (W.R. Grace & Co., Columbia, Md.) catalyst, for example (Ber. (1920) V53 pp 2306, JACS (1923) V45, 3029 and USA 2955133). RANEY® catalysts have a high surface area due to selectively leaching an alloy containing the active metal(s) and a leachable metal (usually aluminum). RANEY® catalysts have high activity due to the higher specific area and allow the use of lower temperatures in hydrogenation reactions. The active metals of RANEY® catalysts include nickel, copper, cobalt, iron, rhodium, ruthenium, rhenium, osmium, iridium, platinum, palladium, compounds thereof and combinations thereof.

Promoter metals may also be added to the base RANEY® metals to affect selectivity and/or activity of the RANEY® catalyst. Promoter metals for RANEY® catalysts may be selected from transition metals from Groups IIIA through VIIIA, IB and IIB of the Periodic Table of the Elements. Examples of promoter metals include chromium, cobalt, molybdenum, platinum, rhodium, ruthenium, osmium, and palladium, typically at about 2% by weight of the total RANEY metal.

The method of using the catalyst to hydrogenate a feed can be performed by various modes of operation generally known in the art. Thus, the overall hydrogenation process can be performed with a fixed bed reactor, various types of agitated slurry reactors, either gas or mechanically agitated, or the like. The hydrogenation process can be operated in either a batch or continuous mode, wherein an aqueous liquid phase containing the precursor to hydrogenate is in contact with gaseous phase containing hydrogen at elevated pressure and the particulate solid catalyst.

Temperature, solvent, catalyst, reactor configuration, pressure and mixing rate are all parameters that affect the hydrogenation. The relationships among these parameters may be adjusted to effect the desired conversion, reaction rate, and selectivity in the reaction of the process.

A preferred temperature is from about 25° C. to 350° C., more preferably from about 100° C. to about 350° C., and most preferred from about 150° C. to 300° C. The hydrogen pressure is preferably about 250-2000 psig, more preferably about 1000-1500 psi.

The reaction may be performed neat, in water or in the presence of an organic solvent. Water is a preferred solvent though others are possible. Useful organic solvents include those known in the art of hydrogenation such as hydrocarbons, ethers, and alcohols. Alcohols are most preferred, particularly lower alkanols, such as methanol and ethanol. The reaction solvent may also be a mixture, as a non-limiting example, mixtures of water and an alcohol. The reaction should be carried out with selectivity in the range of at least 70%. Selectivity of at least 85% is typical. Selectivity is the weight percent of the converted material that is the desired product, where the converted material is the portion of the starting material that participates in the hydrogenation reaction.

Reduction of hexendioates to adipates (FIG. 1, Step F) may also be done biologically using a reductase. A reductase catalyzes the hydrogenation of a carbon-carbon double bond to a carbon-carbon single bond. The hydride source is usually supplied in the form of a reduced nicotinamide cofactor, NADH or NADPH. More specifically, the enzyme catalyzing the adipic acid formation from 2-hexenedioate can be an enoate reductase capable of reducing the carbon-carbon bond in the 2-position near a carboxylate functionality into a carbon-carbon single bond. NADH-dependent fumarate reductase (EC 1.3.1.6) is also a suitable reductase that has been known to catalyze the conversion of fumarate into succinate in the TCA cycle. (A review on E. coli fumarate reductase: Cecchini, G.; Schroder, I.; Gunsalus, R. P.; Maklashina, E. Biochim. Biophys. Acta 2002, 1553, 140) Another enzyme, succinate dehydrogenases (EC 1.3.99.1) can also catalyze the same fumarate to succinate reaction by consuming an equivalent of electron donors, for instances, FAD, cytochrome b, flavin, Fe—S center etc. Enzyme 2-Enoate reductase (EC 1.3.1.31) in Clostridium sp. has been reported to catalyze the NADH-dependent crotonate to butyrate conversion (Buehler, M.; Simon, H. Hoppe-Seyler's, Z. Physiol Chem. 1982, 363, 609). Maleylacetate reductase (EC1.3.1.32) in Cupriavidus necator catalyzes the conversion of 3-oxoadipate to 2-maleylacetate (Seibert, V.; Thiel, M.; Hinner, I. S.; Schlomann, M. Microbiology 2004, 150, 463). Enzymes possessing enoyl reductase activity also exist in fatty acid biosynthesis using enoyl-ACP as substrate may be used. NADH-dependent enoyl-ACP reductase (EC 1.3.1.9) catalyzes the conversion of trans-2-acyl-ACP into acyl-ACP (A review: Massengo-Tiasse, R. P.; Cronan, J. E. Cell Mol. Life. Sci. 2009, 66, 1507).

Adipates and hexenoates may also be converted to mono or di esters prior to reduction to adipic acid. Esterification reactions are well known in the literature (Kirk-Othmer Encyclopedia of Chemical Technology, Vol 10, pages 471-496) and employ homogenous acids such as sulphuric acid and toluenesulfonic acid. Esterifications may also employ heterogeneous acid catalyst such as alumina, zeolites, sulphonic acid resins and sulfonated clays. The mono or diester adipate generated from an ester of hexenedioate can then be converted to adipate or adipic acid.

Direct conversion of 3-hydroxyadipate to adipate or 3-hydroxyadipic acid to adipic acid (Step G in FIG. 1) may be accomplished using a bifunctional catalyst. A heterogeneous catalyst system useful for the reaction is a catalyst system that can function both as an acid catalyst and as a hydrogenation catalyst. The heterogeneous catalyst system can comprise independent catalysts, i.e., at least one solid acid catalyst plus at least one solid hydrogenation catalyst. Alternatively, the heterogeneous catalyst system can comprise a dual function catalyst. For the purposes of this disclosure, a dual function catalyst is a catalyst wherein at least one solid acid catalyst and at least one solid hydrogenation catalyst are combined into one catalytic material.

Suitable acid catalysts for the present methods are heterogeneous (or solid) acid catalysts. The at least one solid acid catalyst may be supported on at least one catalyst support (herein referred to as a “supported acid catalyst”) or may be unsupported (herein referred to as an “unsupported acid catalyst”). Solid acid catalysts include, but are not limited to, (1) heterogeneous heteropolyacids (HPAs) and their salts, (2) natural clay minerals, such as those containing alumina or silica (including zeolites), (3) cation exchange resins, (4) metal oxides, (5) mixed metal oxides, (6) metal salts such as metal sulfides, metal sulfates, metal sulfonates, metal nitrates, metal phosphates, metal phosphonates, metal molybdates, metal tungstates, metal borates, and (7) combinations of groups 1 to 6. When present, the metal components of groups 4 to 6 may be selected from elements from Groups I, IIa, IIIc, VIIa, VIIIa, Ib and IIb of the Periodic Table of the Elements, as well as aluminum, chromium, tin, titanium and zirconium.

Suitable HPAs include compounds of the general Formula Xa MbOcq-, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Non-limiting examples of salts of HPAs are lithium, sodium, potassium, cesium, magnesium, barium, copper, gold and gallium, and onium salts such as ammonia. Methods for preparing HPAs are well known in the art and are described, for example, in Hutchings, G. and Vedrine, J., supra; selected HPAs are also available commercially, for example, through Sigma-Aldrich Corp, (St. Louis, Mo.). Examples of HPAs suitable for the process of this disclosure include tungstosilicic acid (H₄[SiW₁₂O₄₀].xH₂O), tungstophosphoric acid (H₃[PW₁₂O₄₀].xH₂O), molybdophosphoric acid (H₃[PMo₁₂O₄₀].xH₂O), molybdosilicic acid (H₄[SiMo₁₂O₄₀].xH₂O), vanadotungstosilicic acid (H₄₊ₙ[SiVₙW₁₂₋ₙO₄₀].xH₂O), vanadotungstophosphoric acid (H₃+ₙ[PVₙW₁₂₋ₙO₄₀].xH₂O), vanadomolybdophosphoric acid (H₃₊ₙ[PVₙMo₁₂O₄₀].xH₂O), vanadomolybdosilicic acid (H₄₊ₙ[SiVₙMo₁₂₋ₙO₄₀].xH₂O), molybdotungstosilicic acid (H₄[SiMoₙW₁₂₋ₙO₄₀].xH₂O), molybdotungstophosphoric acid (H₃[PMoₙW₁₂₋ₙO₄₀].xH₂O), wherein n in the Formulas is an integer of 1 to 11 and x is an integer of 1 or more.

Natural clay minerals are well known in the art and include, without limitation, kaolinite, bentonite, attapulgite, montmorillonite and zeolites.

Suitable cation exchange resins include styrene-divinylbenzene copolymer-based strong cation exchange resins such as AMBERLYST® (DOW; Philadelphia, Pa.), DOWEX® (for example, DOWEX® Monosphere M-31) (Dow; Midland, Mich.), CG resins from Resintech, Inc. (West Berlin, N.J.), and Lewatit resins such as MonoPlus S 100 H from Sybron Chemicals Inc. (Birmingham, N.J.).

Fluorinated sulfonic acid polymers can also be used as solid acid catalysts for the process of the present disclosure. These acids are partially or totally fluorinated hydrocarbon polymers containing pendant sulfonic acid groups, which may be partially or totally converted to the salt form. One particularly suitable fluorinated sulfonic acid polymer is NAFION® perfluorinated sulfonic acid polymer, (E.I. du Pont de Nemours and Company, Wilmington, Del.). One preferred form is NAFION® Super Acid Catalyst, a bead-form strongly acidic resin which is a copolymer of tetrafluoroethylene and perfluoro-3,6-dioxa-4-methyl-7-octene sulfonyl fluoride, converted to either the proton (H+), or the metal salt form.

Preferred solid acid catalysts include cation exchange resins, such as AMBERLYST® 15 (Rohm and Haas, Philadelphia, Pa.), AMBERLITE® 120 (Rohm and Haas), NAFION®, and natural clay materials, including zeolites such as mordenite.

When used, the at least one support for the at least one solid acid catalyst can be any solid substance that is inert under the reaction conditions including, but not limited to, oxides such as silica, alumina and titania, compounds thereof or combinations thereof; barium sulfate; calcium carbonate; zirconia; carbons, particularly acid washed carbon; and combinations thereof. Acid washed carbon is a carbon that has been washed with an acid, such as nitric acid, sulfuric acid or acetic acid, to remove impurities. The support can be in the form of powder, granules, pellets, or the like. The supported acid catalyst can be prepared by depositing the acid catalyst on the support by any number of methods well known to those skilled in the art of catalysis, such as spraying, soaking or physical mixing, followed by drying, calcination, and if necessary, activation through methods such as reduction or oxidation. The preferred loading of the at least one acid catalyst on the at least one support is from about 0.1 weight percent to about 20 weight percent based on the combined weights of the at least one acid catalyst plus the at least one support, or any amount therebetween.

Examples of supported acid catalysts include, but are not limited to, phosphoric acid on silica, NAFION® on silica, HPAs on silica, sulfated zirconia and sulfated titania.

In preferred examples, the heterogeneous catalyst system converting 3-hydroxyadipate to adipate (Step G) also comprises at least one solid hydrogenation catalyst. The at least one solid hydrogenation catalyst may be supported on at least one catalyst support (herein referred to as a supported hydrogenation catalyst).

The hydrogenation catalyst may be a metal selected from the group consisting of nickel, copper, chromium, cobalt, rhodium, ruthenium, rhenium, osmium, iridium, platinum, palladium, platinum black; compounds thereof; and combinations thereof. It is well-known that Raney-type catalysts may be formed from some of the metals listed above (for example, RANEY nickel® (W.R. Grace & Co., Columbia, Md.)), and these Raney-type catalysts are also expected to be useful as hydrogenation catalysts for the present disclosure. A promoter such as, without limitation, tin, zinc, copper, gold, silver and combinations thereof may be used to affect the reaction, for example, by increasing activity and catalyst lifetime.

Preferred hydrogenation catalysts include ruthenium, iridium, palladium; compounds thereof; and combinations thereof.

The at least one support for the at least one solid hydrogenation catalyst can be any solid substance that is inert under the reaction conditions including, but not limited to, oxides such as silica, alumina and titania; barium sulfate; calcium carbonate; zirconia; carbons, particularly acid washed carbon; and combinations thereof. The catalyst support can be in the form of powder, granules, pellets, or the like. The supported hydrogenation catalyst can be prepared by depositing the hydrogenation catalyst on the support by any number of methods well known to those skilled in the art of catalysis, such as spraying, soaking or physical mixing, followed by drying, calcination, and if necessary, activation through methods such as reduction. The preferred loading of the metal of the at least one solid hydrogenation catalyst on the at least one support is from about 0.1 weight percent to about 20 weight percent based on the combined weights of the metal of the at least one hydrogenation catalyst plus the at least one support.

Preferred supported hydrogenation catalysts include, but are not limited to, ruthenium on carbon, ruthenium on alumina, and iridium on carbon.

Examples of heterogeneous catalyst systems include any unsupported or supported solid acid catalyst as described above with any unsupported or supported hydrogenation catalyst as described above. In a more specific embodiment, the heterogeneous catalyst system can include an unsupported or supported solid acid catalyst wherein the solid acid catalyst is selected from the group consisting of (1) heterogeneous heteropolyacids (HPAs) and their salts, (2) natural clay minerals, such as those containing alumina or silica (including zeolites), (3) cation exchange resins, (4) metal oxides, (5) mixed metal oxides, (6) metal salts such as metal sulfides, metal sulfates, metal sulfonates, metal nitrates, metal phosphates, metal phosphonates, metal molybdates, metal tungstates, metal borates, and (7) combinations of groups 1 to 6, and an unsupported or supported hydrogenation catalyst wherein the hydrogenation catalyst is selected from metals from the group consisting of nickel, copper, chromium, cobalt, rhodium, ruthenium, rhenium, osmium, iridium, platinum, palladium, platinum black; compounds thereof; and combinations thereof, wherein the catalyst support for either the solid acid catalyst and/or the hydrogenation catalyst can be selected from the group consisting of oxides such as silica, alumina and titania; barium sulfate; calcium carbonate; zirconia; carbons, particularly acid washed carbon; and combinations thereof.

In an even more specific example, the heterogeneous catalyst system can include an unsupported or supported solid acid catalyst wherein the solid acid catalyst is selected from the group consisting of cation exchange resins and natural clay minerals, and an unsupported or supported hydrogenation catalyst wherein the hydrogenation catalyst is selected from metals from the group consisting of nickel, copper, chromium, cobalt, rhodium, ruthenium, rhenium, osmium, iridium, platinum, palladium, platinum black, compounds thereof and combinations thereof.

In an even more specific example, the heterogeneous catalyst system can include an unsupported or supported solid acid catalyst wherein the solid acid catalyst is selected from the group consisting of cation exchange resins and natural clay minerals, and an unsupported or supported hydrogenation catalyst wherein the hydrogenation catalyst is selected from metals from the group consisting of ruthenium, iridium, palladium, compounds thereof, and combinations thereof.

The heterogeneous catalyst system can also be a dual function catalyst. Dual function catalysts (also known as bifunctional catalysts) have been reported; for example, Sie, S.T. has described improved catalyst stability using a dual function catalyst to carry out isomerization reactions (Ertl, G., et al (ed) in Handbook of Heterogeneous Catalysis, Volume 4, Section 3.12.4.2 (1997) VCH Verlagsgesellschaft mbH, Weinheim, Germany). In the present disclosure, the dual function catalyst can be a hydrogenation catalyst on an acidic catalyst support. Such dual function catalysts can be prepared in such a way that the catalyst support retains acid functionality after deposition of the hydrogenation catalyst. The dual function catalyst can be prepared by depositing the metal of the hydrogenation catalyst on the acidic catalyst support by any number of methods well known to those skilled in the art of catalysis, such as spraying, soaking or physical mixing, followed by drying, calcination, and if necessary, activation through methods such as reduction. For example, U.S. Pat. No. 6,448,198 (Column 4, line 55 through Column 18, line 9) describes a solid catalyst containing sulfated zirconia and at least one hydrogenating transition metal for use in hydrocarbon transformation reactions (such as isomerization and alkylation), as well as methods for preparing such catalysts. According to one method, the catalyst can be prepared by depositing hydrated zirconia on a catalytic support, calcining the solid, sulfating the solid, depositing a hydrogenating transition metal on the solid, and performing a final calcination of the solid.

A suitable dual function catalyst can be, but is not limited to, a hydrogenation catalyst comprising a metal selected from the group consisting of nickel, copper, chromium, cobalt, rhodium, ruthenium, rhenium, osmium, iridium, platinum, and palladium; compounds thereof; and combinations thereof deposited by any means described above on an acid catalyst selected from the group consisting of (1) heterogeneous heteropolyacids (HPAs) and their salts, (2) natural clay minerals, such as those containing alumina or silica (including zeolites), (3) cation exchange resins, (4) metal oxides, (5) mixed metal oxides, (6) metal salts such as metal sulfides, metal sulfates, metal sulfonates, metal nitrates, metal phosphates, metal phosphonates, metal molybdates, metal tungstates, metal borates, and (7) combinations of groups 1 to 6.

Preferred dual function catalysts include a hydrogenation catalyst comprising a metal selected from the group consisting of nickel, copper, chromium, cobalt, rhodium, ruthenium, rhenium, osmium, iridium, platinum, and palladium; compounds thereof; and combinations thereof deposited by any means described above on an acid catalyst selected from the group consisting of (1) natural clay minerals, such as those containing alumina or silica (including zeolites), (2) cation exchange resins, (3) metal salts such as metal sulfides, metal sulfates, metal sulfonates, metal nitrates, metal phosphates, metal phosphonates, metal molybdates, metal tungstates, metal borates, and (4) combinations of groups 1 to 3.

In addition, dual function catalysts may comprise at least one hydrogenation catalyst on at least one supported acid catalyst. Examples include, but are not limited to, a hydrogenation catalyst comprising a metal selected from the group consisting of nickel, copper, chromium, cobalt, rhodium, ruthenium, rhenium, osmium, iridium, platinum, and palladium; compounds thereof; and combinations thereof deposited by any means described above on sulfated titania, sulfated zirconia, phosphoric acid on silica, and NAFION® on silica. In a more specific embodiment, platinum can be deposited by any means described above on sulfated titania, sulfated zirconia, phosphoric acid on silica, HPAs on silica, or NAFION® on silica.

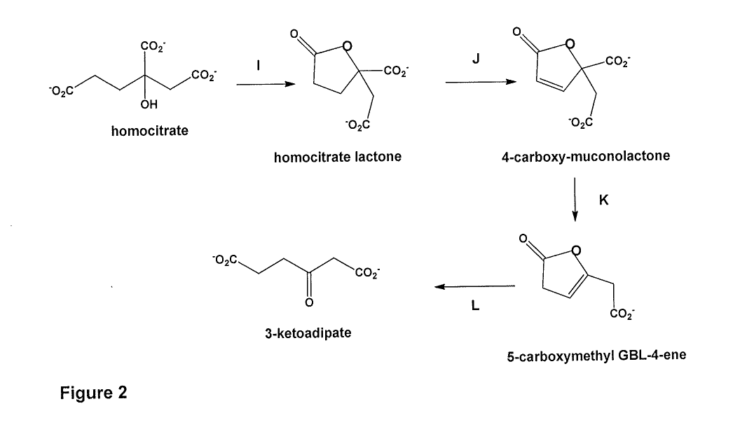

Further examples include chemical transformation to 3-ketoadipate from homocitric acid lactone (Steps J-L in FIG. 2). 3-ketoadipate can be transformed into adipic acid catalytically, for example, according to the pathways shown in FIG. 1 and described above. The pathway shown in FIG. 2 couples the homocitrate biosynthesis and chemical catalysis of homocitric acid lactone to form 3-ketoadipic acid. The dehydrogenation of homocitric acid lactone (Step J, FIG. 2) is selective and leads to formation of the key intermediate 4-carboxymuconolactone. Dehydrogenation of lactones, especially complex multi-cyclic types is a known reaction. This may be accomplished via oxidative routes (DR Buckel and IL Pinto in Chapt 2.2 Oxidation adjacent to C═X bonds and references 121,128,129, 130 and 131 therein in Comprehensive Organic Synthesis, Volume 7, BM Trost, Ed., (1991), Pergammon Press and J. Chem. Soc., Perkin Trans. 1, 1982, 1919-1922 and Chem Commun., (2011), 47(33), 9495 and a paper by RP Dutta and HH Schobert (PSU Fuel Science) accessible at http://web.anl.gov/PCS/acsfuel/preprint %20archive/Files/40_{—}4_CHICAGO_{—}08-95_{—}0950.pdf and J. Chem. Soc. C, 1967, 1720) using DDQ, benzeneseleninic anhydride or metal oxides such as MnO₂ and NiO₂ or Molybdenum based catalysts. Alternately palladium or platinum on carbon or alumina in a high boiling solvent may be employed (The Catalytic Reaction Guide, (2007), Johnson Matthey Catalysts.). High boiling solvents that may be used include p-cymene, diglyme and tetraglyme, high MW aliphatic hydrocarbon oils, naphthalene, durene and decalin.

Further catalytic conversion of adipic acid produced by any of the above pathways can produce other compounds including but not limited to hexamethylene (HMDA), adiponitrile (ADN), caprolactam (CL), Nylon 6 and Nylon 6.6.

It should be understood that chemical compounds referred to herein include acids and salts thereof. Furthermore, it should be understood that reference to an acid form of a compound may be used interchangeably with the salt form.

Additionally, it should be understood that the microorganisms may be modified to express or not express proteins, including those disclosed in U.S. Pat. No. 8,133,704, incorporated herein by reference in its entirety, such as proteins the play a role in aiding the production of compounds of interest by fermentation or carbon sources.

All patents, published patent applications, publications and the subject matter mentioned therein are incorporated herein by reference. The publications discussed herein are provided solely for their disclosure prior to the filing date of this disclosure. Nothing herein is to be construed as an admission that this application is not entitled to antedate such publication by virtue of prior invention.

Although our processes have been described in connection with specific steps and forms thereof; it will be appreciated that a wide variety of equivalents may be substituted for the specified elements and steps described herein without departing from the spirit and scope of this disclosure as described in the appended claims.

### EXAMPLES

The materials used in the following Examples were as follows: Recombinant DNA manipulations generally followed methods described by Sambrook et al. Molecular Cloning: A Laboratory Manual, Third Edition, Sambrook and Russell, 2001, Cold Spring Harbor Laboratory Press, 3rd Edition. Restriction enzymes were purchased from New England Biolabs (NEB). T4 DNA ligase was obtained from Invitrogen. FAST-LINK™ DNA Ligation Kit was obtained from Epicentre. Zymoclean Gel DNA Recovery Kit and DNA Clean & Concentrator Kit was obtained from Zymo Research Company. Maxi and Midi Plasmid Purification Kits were obtained from Qiagen. Antarctic phosphatase was obtained from NEB. Agarose (electrophoresis grade) was obtained from Invitrogen. TE buffer contained 10 mM Tris-HCl (pH 8.0) and 1 mM Na2EDTA (pH 8.0). TAE buffer contained 40 mM Tris-acetate (pH 8.0) and 2 mM Na2EDTA.

In Examples 1-2, restriction enzyme digests were performed in buffers provided by NEB. A typical restriction enzyme digest contained 0.8 μg of DNA in 8 μL of TE, 2 μL of restriction enzyme buffer (10× concentration), 1 μL of bovine serum albumin (0.1 mg/mL), 1 μL of restriction enzyme and 8 μL TE. Reactions were incubated at 37° C. for 1 h and analyzed by agarose gel electrophoresis. When DNA was required for cloning experiments, the digest was terminated by heating at 70° C. for 15 min followed by extraction of the DNA using Zymoclean gel DNA recovery kit.

The concentration of DNA in the sample was determined as follows. An aliquot (10 μL) of DNA was diluted to 1 mL in TE and the absorbance at 260 nm was measured relative to the absorbance of TE. The DNA concentration was calculated based on the fact that the absorbance at 260 nm of 50 μg/mL of double stranded DNA is 1.0.

Agarose gel typically contained 0.7% agarose (w/v) in TAE buffer. Ethidium bromide (0.5 μg/ml) was added to the agarose to allow visualization of DNA fragments under a UV lamp, Agarose gel was run in TAE buffer. The size of the DNA fragments were determined using two sets of 1 kb Plus DNA Ladder obtained from Invitrogen.

### Example 1

### Cloning of Plasmid pBA006

Plasmid pETDuet-nifV-aksF_Mb was constructed from base vector pETDuet1 (Novagen) engineered to include the E. coli codon-optimized homocitrate synthase (nifV) from Azotobacter vinelandii encoded by the sequence shown in SEQ ID NO: 2 and homoisocitrate dehydrogenase (aksF_Mb) from Methanosarcina barkerii shown in SEQ ID NO: 3.

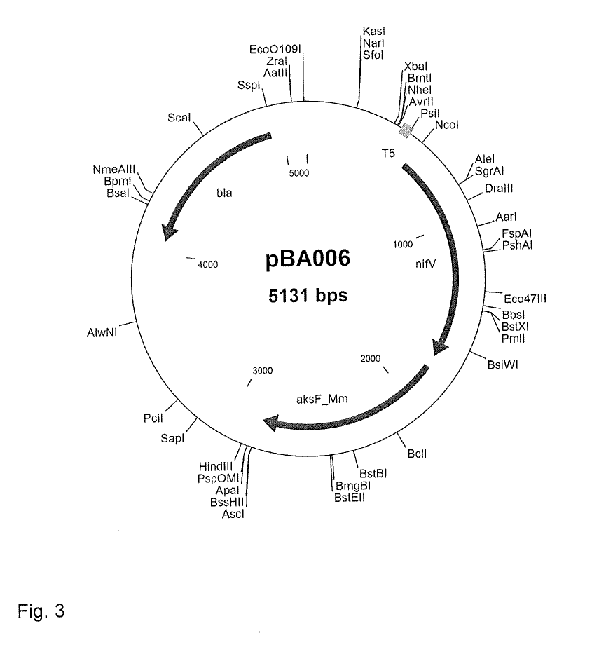

Plasmid pBA001 was constructed from base vector pUC57 to include the T5 promoter region according to SEQ ID NO: 4 and the E. coli codon-optimized homoisocitrate dehydrogenase (aksF_Mm) from Methanococcus maripaludis shown in SEQ ID NO: 5, The DNA fragment containing the nifV ORF was amplified from pETDuet-nifV-aksF_Mb by PCR using primers KL021 (SEQ ID NO: 6) and KL022 (SEQ ID NO: 7). The resulting 1.2 kb DNA was digested with NcoI and EcoNI. The 4.0 kb DNA fragment containing the pUC57 plasmid backbone, T5 promoter region, and aksF_Mm genes was obtained by restriction enzyme digestion of pBA001 using NcoI and EcoNI. The two DNA fragments were ligated to produce plasmid pBA006, as shown by schematic diagram in FIG. 3.

### Example 2

### Cloning of Plasmid pBA066

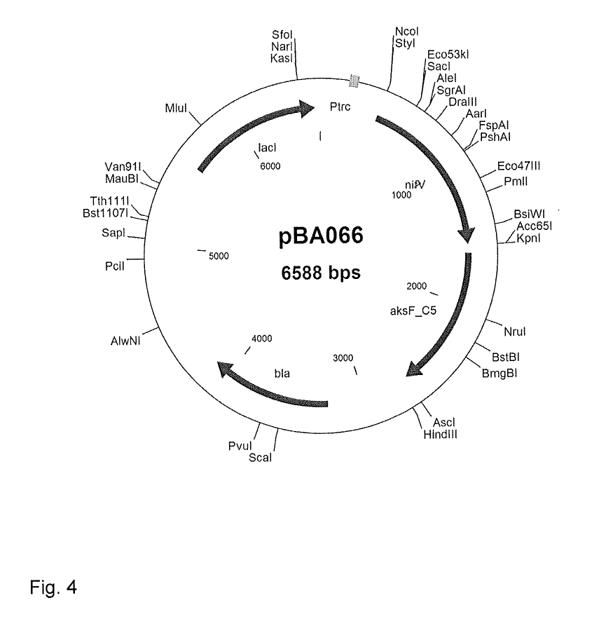

The DNA fragment containing the nifV-aksF_Mm genes was excised from plasmid pBA006 using NcoI and HindIII. The fragment was then ligated to the pTrcHisA (Invitrogen), which had been digested with NcoI and HindIII, to produce pBA066, as shown by schematic diagram in FIG. 4.

### Example 3

Circular plasmid DNA molecules were introduced into target E. coli cells by chemical transformation or electroporation. For chemical transformation, cells were grown to mid-log growth phase, as determined by the optical density at 600 nm (0.5-0.8). The cells were harvested, washed and finally treated with CaCl₂. To chemically transform these E. coli cells, purified plasmid DNA was allowed to mix with the cell suspension in a microcentrifuge tube on ice. A heat shock was applied to the mixture and followed by a 30-60 min recovery incubation in rich culture medium. For electroporation, E. coli cells grown to mid-log growth phase were washed with water several times and finally resuspended into 10% glycerol solution. To electroporate DNA into these cells, a mixture of cells and DNA was pipetted into a disposable plastic cuvette containing electrodes. A short electric pulse was then applied to the cells which to form small holes in the membrane where DNA could enter. The cell suspension was then incubated with rich liquid medium followed by plating on solid agar plates. Detailed protocol could be obtained in Molecular Cloning: A Laboratory Manual, Third Edition, Sambrook and Russell, 2001, Cold Spring Harbor Laboratory Press, 3rd Edition.

E. coli cells of the BL21 strain were transformed with plasmid pBA066. BL21 is a strain of E. coli having the genotype: B F- dcm ompT hsdS(rB- mB-) gal λ. BL21 transformant of pBA066 is also called biocatalyst BA066.

### Example 4

### Cell Lysis Method

E. coli cell culture was spun down by centrifugation at 4000 rpm. The cell-free supernatant was discarded and the cell pellet was collected. After being collected and resuspended in the proper resuspension buffer (50 mM phosphate buffer at pH 7.5), the cells were disrupted by chemical lysis using BUGBUSTER® reagent (Novagen). Cellular debris was removed from the lysate by centrifugation (48,000 g, 20 min, 4° C.). Protein was quantified using the Bradford dye-binding procedure. A standard curve was prepared using bovine serum albumin. Protein assay solution was purchased from Bio-Rad and used as described by the manufacturer.

### Example 5

### Homocitrate Synthase Activity in BA066 Crude Lysate

High-throughput in vitro homocitrate synthase activity was assayed in a 96-well plate format to verify expression and activity of homocitrate synthase (NifV) in BL21 cells transformed with plasmid pBA042. The assay protocol was modified from a literature procedure (Zheng, L.; White, R. H.; Dean, D. R. J. Bacteriol. 1997, 179, 5963).

A typical assay mixture was composed of 20 mM a-ketoglutarate and 0.2 mM acetyl CoA, 5 mM MgSO4 and 1 mM DTNB (5,5′-dithiobis(2-nitrobenzoic acid)) in 10 mM Tris buffer at pH 8 to a total volume of 200 μL per well.

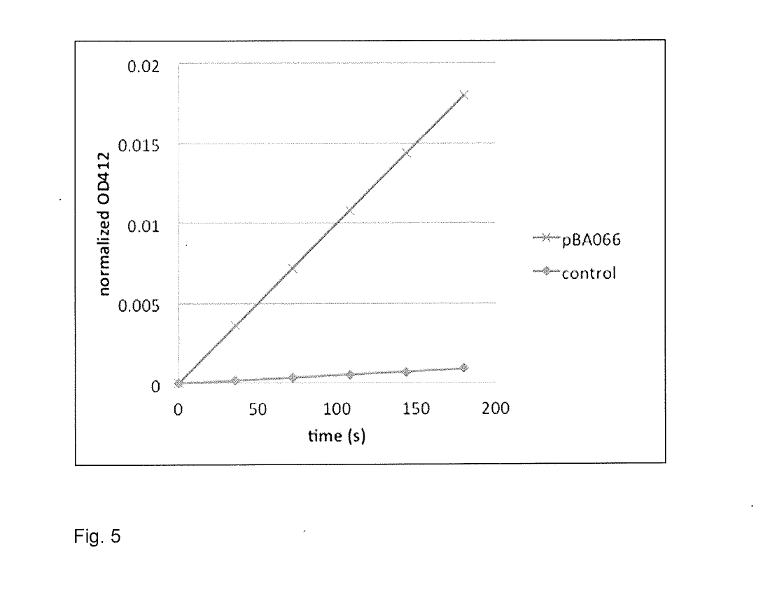

The assay was initiated by the addition of a 20 uL of cell lysate and was followed spectrophotometrically by monitoring color change at 412 nm. A unit of activity equals 1 μmol per min of homocitrate formed at 30° C. As shown in FIG. 5, BL21 control lysate showed negligible background activity. Crude lysate of BL066 showed activity at around 0.017 U/mg under the same conditions.

### Example 6

### SDS-PAGE Analysis of Homocitrate Synthase Expression

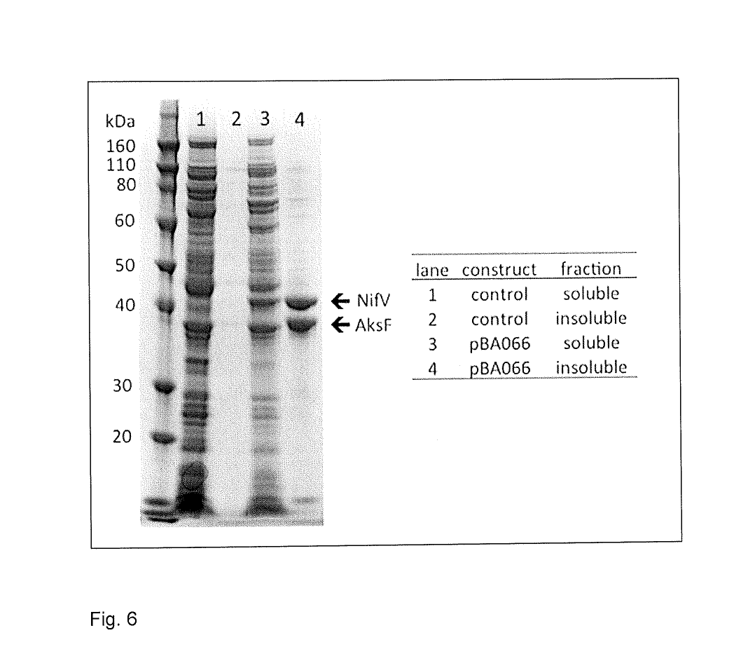

SDS-PAGE was used to analyze protein expression in constructs BL21/pTrcHisA (control) and BA066 (FIG. 6). Lanes 1 and 2 are samples of solution and the insoluble fraction of the control construct, respectively. Lanes 3 and 4 are samples of solution and the insoluble fraction of the BA066 construct, respectively.

The molecular weight of the nifV encoding homocitrate synthase is 42 kDa, while the aksF gene encodes isohomocitrate dehydrogenase of 38 kDa. As shown in FIG. 6, proteins having the same molecular weight as NifV and AksF were successfully expressed.

### Growth Medium

For the following Examples, Examples 7-8, the Growth Medium was prepared as follows:

All solutions were prepared in distilled, deionized water. LB medium (1 L) contained Bacto tryptone (i.e. enzymatic digest of casein) (10 g), Bacto yeast extract (i.e. water soluble portion of autolyzed yeast cell) (5 g), and NaCl (10 g). LB-glucose medium contained glucose (10 g), MgSO4 (0.12 g), and thiamine hydrochloride (0.001 g) in 1 L of LB medium. LB-freeze buffer contained K₂HPO₄ (6.3 g), KH₂PO₄ (1.8 g), MgSO₄ (1.0 g), (NH₄)₂SO₄ (0.9 g), sodium citrate dihydrate (0.5 g) and glycerol (44 mL) in 1 L of LB medium. M9 salts (1 L) contained Na₂HPO₄ (6 g), KH₂PO₄ (3 g), NH₄Cl (1 g), and NaCl (0.5 g). M9 minimal medium contained D-glucose (10 g), MgSO₄ (0.12 g), and thiamine hydrochloride (0.001 g) in 1 L of M9 salts. Antibiotics were added where appropriate to the following final concentrations: ampicillin (Ap), 50 μg/mL; chloramphenicol (Cm), 20 μg/mL; kanamycin (Kan), 50 μg/mL; tetracycline (Tc), 12.5 μg/mL. Stock solutions of antibiotics were prepared in water with the exceptions of chloramphenicol which was prepared in 95% ethanol and tetracycline which was prepared in 50% aqueous ethanol. Aqueous stock solutions of isopropyl-β-D-thiogalactopyranoside (IPTG) were prepared at various concentrations.

The standard fermentation medium (1 L) contained K₂HPO₄ (7.5 g), ammonium iron (III) citrate (0.3 g), citric acid monohydrate (2.1 g), and concentrated H₂SO₄ (1.2 mL). Fermentation medium was adjusted to pH 7.0 by addition of concentrated NH₄OH before autoclaving. The following supplements were added immediately prior to initiation of the fermentation: D-glucose, MgSO₄ (0.24 g), potassium and trace minerals including (NH₄)6(Mo₇O₂₄).4H2O (0.0037 g), ZnSO₄.7H₂O (0.0029 g), H₃BO₃ (0.0247 g), CuSO₄.5H₂O (0.0025 g), and MnCl₂.4H₂O (0.0158 g). IPTG stock solution was added as necessary (e.g., when optical density at 600 nm lies between 15-20) to the indicated final concentration. Glucose feed solution and MgSO4 (1 M) solution were autoclaved separately. Glucose feed solution (650 g/L) was prepared by combining 300 g of glucose and 280 mL of H₂O. Solutions of trace minerals and IPTG were sterilized through 0.22-μm membranes. Antifoam (Sigma 204) was added to the fermentation broth as needed.

### Example 7

### Shake Flask Experiments for Homocitrate Production

Seed inoculant was started by introducing a single colony of biocatalyst BA066 picked from a LB agar plate into 50 mL TB medium (1.2% w/v bacto Tryptone, 2.4% w/v Bacto Yeast Extract, 0.4% v/v glycerol, 0.017 M KH₂PO₄, 0.072 M K₂HPO₄). Culture was grown overnight at 37° C. with agitation at 250 rpm until they were turbid. A 2.5 mL aliquot of this culture was subsequently transferred to 50 mL of fresh TB medium. After culturing at 37° C. and 250 rpm for an additional 3 h, IPTG was added to a final concentration of 0.2 mM. The resulting culture was allowed to grow at 27° C. for 4 hours. Cells were harvested, washed twice with PBS medium, and resuspended in 0.5 original volume of M9 medium supplemented with glucose (2 g/L). The whole cell suspension was then incubated at 27° C. for 48 h. Samples were taken and analyzed by GC/MS and ¹H-NMR. Compared to the control BL21 strain transformed with empty plasmids, E. coli BA066 produced homocitrate at a concentration of 0.5 g/L in shake flasks from glucose.

### Example 8

### Cultivation of Homocitrate Biocatalyst Under Fermentor-Controlled Conditions

Fed-batch fermentation was performed in a 2 L working capacity fermentor. Temperature, pH and dissolved oxygen were controlled by PID control loops. Temperature was maintained at 37° C. by temperature adjusted water flow through a jacket surrounding the fermentor vessel at the growth phase, and later adjusted to 27° C. when production phase started. The pH was maintained at 7.0 by the addition of 5 N KOH and 3 N H₃PO₄. Dissolved oxygen (DO) level was maintained at 20% of air saturation by adjusting air feed as well as agitation speed.

Inoculant was started by introducing a single colony of BA066 picked from an LB agar plate into 50 mL TB medium. The culture was grown at 37° C. with agitation at 250 rpm until the medium was turbid. Subsequently a 100 mL seed culture was transferred to fresh M9 glucose medium. After culturing at 37° C. and 250 rpm for an additional 10 h, an aliquot (50 mL) of the inoculant (OD600=6-8) was transferred into the fermentation vessel and the batch fermentation was initiated. The initial glucose concentration in the fermentation medium was about 40 g/L.

Cultivation under fermentor-controlled conditions was divided into two stages. In the first stage, the airflow was kept at 300 ccm and the impeller speed was increased from 100 to 1000 rpm to maintain the DO at 20%. Once the impeller speed reached its preset maximum at 1000 rpm, the mass flow controller started to maintain the DO by oxygen supplementation from 0 to 100% of pure O₂.

The initial batch of glucose was depleted in about 12 hours and glucose feed (650 g/L) was started to maintain glucose concentration in the vessel at 5-20 g/L. At OD600=20-25, IPTG stock solution was added to the culture medium to a final concentration of 0.2 mM. The temperature setting was decreased from 37 to 27° C. and the production stage (i.e., second stage) was initiated. Production stage fermentation was run for 48 hours and samples were removed to determine the cell density and quantify metabolites.

The homocitrate production was measured by GS/MS and ¹H-NMR. Compared to the control BL21 strain transformed with empty plasmids, E. coli BA066 produced homocitrate from glucose at a concentration of 2 g/L under fermentor-controlled conditions.

The following examples describe the preparation of adipates or adipic acid from 2-ketoglutarate.

### Example 9

### Chemical Conversion of Homocitrate to 3-Hydroxyadipate (B′)

A 5-50% weight % solution of homocitrate is contacted with aqueous sulfuric acid solution of a concentration of 3% to 50% and at temperatures in the range of 50-200° C. (atmospheric or super-atmospheric pressure) for 30 minutes to 5 hrs with good stirring during which time CO₂ is evolved and 3-hydroxyl adipate is formed. The ratio of sulfuric acid to homocitrate is in the range of 0.5 moles to 10 moles of sulfuric acid to 1 mole of homocitrate, preferably 0.5 moles to 2 moles sulfuric acid to 1 mole of homocitrate.

### Example 10

### Oxidative Decarboxylation of Homocitrate to 3-Hydroxyadipate (B′)

A 50 wt. % aqueous solution of homocitrate is contacted with catalyst containing copper or copper ions, of a porphyrin or EDTA complex, and an oxidizing agent such as hydrogen peroxide, mono-peroxy sulfate or O₂ (at 1-20 atmospheres) and heated to 30-100° C. for 2-10 hours with good stirring. 3-Hydroxyadipate is the major product identified by gas chromatography.

### Example 11

### Photochemical Decarboxylation of Homocitrate to 3-Hydroxyadipate (B′)

To 500 ml of 10% aqueous solution of homocitrate is added to 10,000 mg of TiO₂ powder to form a slurry and exposed to light in a quartz vessel for 24 hrs at 25° C. 3-Hydroxyadipate and carbon dioxide are the major products formed. Recovery of 3-hydroxyadipate is easily accomplished by removing the TiO₂ catalyst via filtration and evaporation of the water of solution.

### Example 12

### Chemical Dehydration of 3-Hydroxyadipate to Hexenedioate (E)

A 40% aqueous solution of homocitrate is contacted with either a solution of a Lewis acid component such as aluminum sulfate or a solid Lewis acid such as a silica-alumina or tungstated zirconia and heated. The desired temperatures are in the range of 50-200° C. (atmospheric or super-atmospheric pressure) for 30 minutes to 5 hrs. during which time CO₂ is evolved and 3-hydroxyl adipate is formed. The ratio of Lewis acid to homocitrate is in the range of 0.5 moles to 20 moles, preferably 0.5 moles to 5 moles Lewis acid to 1 mole of homocitrate.

### Example 13

### Decarboxylation of Homocitrate to 3-Ketoadipate (C′)

A solution of 300 ml of a 50% by weight solution of homocitrate in water is placed in a 500 ml. autoclave and combined with 10 grams of a pre-reduced 1% platinum supported on silica-alumina catalyst. After purging and sealing the autoclave it is heated to 300° C. and held at that temperature for 2 hours with good stirring. After the reaction time is completed, the autoclave is cooled and the contents withdrawn. 3-Ketoadipate is recovered in near quantitative yield from the reactor product solution along with minor amounts of unidentified components.

### Example 14

### Decarboxylation of Homocitrate to 3-Ketoadipate (C′)

A 16″ long×0.5″ ID 316 SS diameter tubular reactor is loaded with 25 cc of 5% palladium supported on granular carbon. Ten cc of SS balls 1/16″ diameter are loaded under the Pd/C catalyst and also above it to act as a bed support and preheat zones respectively. The catalyst is activated by passing hydrogen gas at a flow rate of 25 cc/min through the reactor while heating at a rate of 2° C./minute to 300° C. at 1 atmosphere pressure. The hydrogen gas flow is continued at 300° C. for 1 hour and then the gas flow is switched to helium at a flow rate of 10 cc/min and the reactor pressure raised to 3 atm by use of a back pressure control valve. When the temperature and pressure stabilized, a 50% by weight solution of homocitrate in water is pumped into the reactor at a flow rate of 10 cc/minute and this flow continued until 500 cc of product solution is collected downstream of the back pressure control valve. Helium was flowed concurrently with the homocitrate solution during the run. Greater than 90% of the theoretical yield of 3-ketoadipate is recovered from the solution along with minor amounts of 3-hydroxyadipate and other unidentified components.

### Example 15

### Decarboxylation of Homocitric Acid to 3-Ketoadipic Acid (C′)

The above Example 14 is repeated exactly but with the substitution of the water as solvent with dioxane and the use of homocitric acid. In this case, greater than about 75% of the theoretical yield of 3-ketoadipate is recovered from the reaction product solution along with minor amounts of 3-hydroxyadipate and other unidentified components.

### Example 16

### Oxidative Decarboxylation of Homocitrate to 3-Ketoadipate (C′)

In this example of oxidative decarboxylation, 300 ml of a 50% by weight solution of homocitrate in water is placed in a 500 ml autoclave and combined with 10 grams of a mixed oxide catalyst composed of tin, bismuth and molybdenum oxides which had been prepared via co-precipitation and calcination at 500° C. After purging and sealing the autoclave, it was pressured to 500 psig with air then heated to 250° C. and held at that temperature for 2 hours with good stirring. After the reaction time is complete, the autoclave is cooled and the contents withdrawn. 3-Keto adipate is recovered in near quantitative yield from the reactor product solution along with minor amounts of unidentified components.

### Example 17

### Oxidative Decarboxylation of Homocitrate to 3-Ketoadipate (C′)

In this case, Example 16 above is repeated with the exception that no air pressure is employed. The reaction temperature is 150° C. and 150 ml of 30% hydrogen peroxide is pumped slowly into the reactor at the reaction temperature. The time of addition of the H₂O₂ is 1 hour and the reactor is held at 150° C. for an additional hour after the H₂O₂ addition is completed. After the reaction time is complete, the autoclave is cooled and the contents withdrawn. 3-keto adipate is recovered in near quantitative yield from the reactor solution along with minor amounts of unidentified components.

### Example 18

### Hydrogenation of 3-Ketoadipate to 3-Hydroxyadipate

An aqueous solution of 3-ketoadipate, 40% by weight in water and 300 ml of total volume is placed into a 500 ml autoclave along with a 5% ruthenium on carbon catalyst which has been obtained in pre-reduced form. After purging the reactor to remove air, it is pressurized to 850 psig with hydrogen gas and heated with good stirring to 80° C. while maintaining a hydrogen pressure of 850 psig. These conditions are maintained for 6 hours after which time the autoclave is cooled, the contents filtered to remove the catalyst and a near theoretical yield of 3 hydroxyadipate is recovered along with minor amounts of adipate and small amounts of unidentified materials.

### Example 19

### Hydrogenation of 3-Hexenoate to Adipate

300 ml of a 30% solution of hexenedioate in dioxane, is added to a 500 ml batch autoclave reactor with 2 grams of 1% Pt on 1 mm diameter gamma alumina particles. After purging to remove air and sealing the reactor, it is pressurized with hydrogen gas to 800 psig while the reactor is heated to 80° C. Temperature and pressure are maintained for 4 hours with good stirring. At the end of the reaction period, the pressure is released and adipate is recovered from the reaction mass product by conventional means yielding greater than 90% of the theoretical amount.

### Example 20

### Hydrogenation of 3-Hexenedioic Acid to Adipic Acid

300 ml of a 30% solution of hexenedioic acid in water is added to a 500 ml batch autoclave reactor with 5 grams (dry weight) of water-wet RANEY® nickel catalyst. After purging to remove air and sealing the reactor, it is pressurized continuously with hydrogen gas to 1200 ping while the reactor is heated to 140° C. for 4 hours with good stirring. At the end of the reaction period, the pressure is released and adipic acid is recovered from the reaction mass product by conventional means yielding greater than 90% of the theoretical amount.

### Example 21

### Hydrogenation of Diethyl-3-Ketoadipate to Diethyladipate

300 Ml of a 30 wt % solution of diethyl-3-ketoadipate in ethanol is added to a 500 ml autoclave containing 5 g of Ru/C catalyst. The reactor is heated to 250° C. under 1500 psi of hydrogen and maintained at temperature and pressure for 4 hours. At the end of the reaction, the vessel is cooled and diethyladipate is recovered in >90% yield by distillation.

### Example 22

### Dehydrogenation of Homocitrate Lactone to 4-Carboxymethyl GBL-4-Ene (Steps J and K, FIG. 2)

A solution of homocitric lactone of 20% by weight in diglyme and volume of 250 ml is placed into a 500 ml round bottom flask equipped with a flowing tap water cooled condenser and a large magnetic stir bar. To this solution is added 5 grams of 10% Pd/C catalyst which has been obtained in the pre-reduced form. The stirrer is activated and the slurry heated to reflux for 16 hours. At the end of the reaction period, the reaction mass is cooled and a greater than 90% of 4-carboxy-muconolactone theoretical yield of is obtained along with minor amounts of 5-carboxy-methyl GBL-4-ene. This demonstrates dehydrogenation and subsequent decarboxylation in a single pot.

### Example 23

### Oxidative Dehydrogenation of Homocitrate Lactone to 4-Carboxy-Muconolactone and 5-Carboxmethyl GBL-4-Ene (Steps J and K of FIG. 2) Using DDQ

DDQ (2,3-Dichloro-5,6-dicyano-1,4-benzoquinone is used as an oxidative dehydrogenation reagent. A solution of 18.6 grams homocitric lactone in 600 ml of water is placed into a 1000 ml round bottom flask equipped with a flowing tap water cooled condenser and a large magnetic stir bar. To this is added 0.6 moles of DDQ (136.2 grams) and the flask is heated to 40° C. for 16 hours with good stirring. At the end of the reaction period, the reaction mass is cooled and a greater than 90% of theoretical yield of combined amounts of 5-carboxymethyl GBL-4-ene and 4-carboxy-muconolactone along with minor amounts of unidentified materials is obtained.

### Example 24

### Oxidative Dehydrogenation of Homocitrate Lactone to 4-Carboxy-Muconolactone and 5-Carboxmethyl GBL-4-Ene (Steps J and K of FIG. 2) Using and Oxidation Catalyst

A solution of homocitric lactone of 20% by weight in diglyme and volume of 250 ml is placed into a 500 ml pressure autoclave. Into this solution is placed 10 grams of previously prepared molybdenum based catalyst prepared using ammonium tetra thiomolybdate (ATTM) as Mo precursor. The catalyst was prepared by hydrogenation of ATTM under 1100 psig Hydrogen pressure for 6 hours at 400° C. as described by RP Dutta and HH Schobert (and J. Chem. Soc. C, 1967, 1720). The reactor is purged to remove air and replace the gas atmosphere with nitrogen and then the autoclave is sealed and heated to 300° C. for 1 hour with good stirring. After the end of the reaction heating period, the reaction mass is cooled and a greater than 90% of theoretical yield of combined amounts of 5-carboxymethyl GBL-4-ene and 4-carboxy-muconolactone along with minor amounts of unidentified materials is obtained again demonstrating dehydrogenation and subsequent decarboxylation in a single pot.

### Example 25

### Path “H” Direct Hydrogenolysis of 3-Ketoadipate to Adipic

A 100 mls batch autoclave is loaded with 0.0.2 grams of IrCl₃, 1.5 grams of LiI, 1.5 cc or 50% HI, 5 cc DI water, 30 cc of Acetic acid and 35 grams of 3 keto adipate. The reactor is sealed, purged with He to remove air and then pressured with 285 psig of Carbon Monoxide and 520 psig of hydrogen (total pressure of 805 psig) and heated to 190° C. with good stirring. Heating and stirring is maintained for 20 hrs while additional hydrogen gas is added so as to maintain the total pressure constant at 805 psig. At the end of the reaction period, the reactor is vented to reduce pressure to atmospheric and cooled to room temperature. Analysis of the product indicates the keto adipate is converted substantially to adipate.

### Example 26

### Direct Hydrogenolysis of 3-Ketoadipate to Adipic Acid

This above example is repeated exactly with the exceptions that the IrCl3 was replaced with a like amount of RhC13 and the solvent is changed to an equal volume mixture of propionic acid and acetic acid (15 mls each of acetic and propionic acids). At end of the reaction period, analysis of the product indicates the keto adipate is converted substantially to adipate.

1. A method for producing adipate or adipic acid comprising:
a) condensing 2-ketoglutaric acid or salt thereof with acetyl-CoA to form homocitric acid or a salt thereof;
b) converting homocitric acid or a salt thereof to adipate or adipic acid by at least one chemical reaction; and
c) optionally, isolating adipate or adipic acid.

9. The process of claim 1, further comprising a heterogeneous catalyst system, wherein the heterogeneous catalyst system comprises:
a) at least one unsupported or supported solid acid catalyst wherein the solid acid catalyst is selected from the group consisting of (1) heterogeneous heteropolyacids and their salts, (2) natural clay minerals, (3) cation exchange resins, (4) metal oxides, (5) mixed metal oxides, (6) metal salts and (7) combinations of groups 1 to 6; and
b) at least one unsupported or supported hydrogenation catalyst wherein the hydrogenation catalyst is selected from metals from the group consisting of nickel, copper, chromium, cobalt, rhodium, ruthenium, rhenium, osmium, iridium, platinum, palladium, platinum black, compounds thereof, and combinations thereof.

12. The method of claim 1, wherein the chemical catalyst comprises:
a) at least one unsupported or supported solid acid catalyst wherein the solid acid catalyst is selected from the group consisting of (1) heterogeneous heterogeneous and their salts, (2) natural clay minerals, (3) cation exchange resins, (4) metal oxides, (5) mixed metals oxides, (6) metal salts and (7) combinations thereof; and
b) at least one unsupported or supported hydrogenation catalyst wherein the hydrogenation catalyst is selected from metals from the group consisting of nickel, copper, chromium, cobalt, rhodium, ruthenium, rhenium, osmium, iridium, platinum, palladium, platinum black; compounds thereof; and combinations thereof.

13. The method of claim 1, wherein the chemical catalyst is unsupported or supported.

14. The method of claim 1, where in the chemical catalyst is at least one selected from a heterogeneous catalyst, a homogeneous catalyst, a dual catalyst and a hydrogenation catalyst.

15. The method of claim 1, wherein the chemical catalyst comprises a solid acid catalyst selected from the group consisting of cation exchange resin and natural clay materials.

16. The method of claim 1, wherein the chemical catalyst comprises a hydrogenation catalyst selected from the group consisting of nickel, copper, chromium, cobalt, rhodium, ruthenium, rhenium, osmium, iridium, platinum, palladium, platinum black, compounds thereof, and combinations thereof.

23. The method of claim 1, wherein the chemical catalyst is contacted with the homocitric acid at a temperature between about 75 and about 300° C. and a hydrogen pressure between about 345 kPA and about 20.7 MPa.

24. The method of claim 1, further comprising isolating adipate or adipic acid.

25. The method of claim 1, wherein the at least one chemical conversion comprises at least one of the product to substrate conversion selected from 1) hexenedioic acid or a salt thereof, 2) 3-ketoadipic acid or a salt thereof and 3) 3-hydroxyadipic acid or a salt thereof, to adipic acid or a salt thereof.
